(19) United States
(12) Patent Application Publication
(10) Pub. No.: US 2015/0321992 A1
(43) Pub. Date: Nov. 12, 2015

(54) TRIACYLGLYCEROL OLIGOMERS

(71) Applicant: Trent University, Peterborough (CA)

(72) Inventors: Suresh Narine, Peterborough (CA); Shaojun Li, Peterborough (CA); Laziz Bouzidi, Peterborough (CA)

(73) Assignee: Trent University, Peterborough (CA)

(21) Appl. No.: 14/703,986

(22) Filed: May 5, 2015

Related U.S. Application Data
(60) Provisional application No. 61/989,722, filed on May 7, 2014.

(51) Int. Cl. C07C 69/73

(52) U.S. Cl. CPC C07C 69/73

(57) ABSTRACT

This application relates to triacylglycerol oligomers derived from the metathesis of natural oils. These oligomers are structure controlled dimers and quatrimers, and the effect of saturation, molecular size, and positional isomerization are also described herein.

### CROSS REFERENCE TO RELATED APPLICATIONS

A claim of priority for this application under 35 U.S.C. §119(e) is hereby made to U.S. Provisional Patent Application No. 61/989,722, filed May 7, 2014, which is hereby incorporated by reference in its entirety.

### TECHNICAL FIELD

This application relates to triacylglycerol oligomers derived from the metathesis of natural oils. These oligomers are structure controlled dimers and quatrimers, and the effect of saturation, molecular size, and positional isomerization are also described.

### BACKGROUND

Oligomers of triacylglycerols may be derived from the metathesis of natural oils. Such oligomers are often sought for a variety of end-use applications, which include but are not limited to, biobased waxes, base stocks for lubricant applications or a base stock blend component for use in a finished lubricant, and crystallization depressant additives and/or crystal size reduction additives for biodiesel.

Due to the complex composition of oligomerized metathesis products, the isolation of individual components that may serve to facilitate the structure-function relationships of these materials is often difficult. However, knowledge of these relationships is of vital importance for designing product compositions that deliver functionality required in commercial products. One approach is to synthesize the individual components and use them as model systems to understand their individual and composite effects on the properties of the metathesized materials. The effect of size on the crystallization, melting and flow behaviors of such TAG oligomers has been previously investigated using model compounds by Li, S., L. Bouzidi, and S. S. Narine, Synthesis and Physical Properties of Triacylglycerol Oligomers: Examining the Physical Functionality Potential of Self-Metathesized Highly Unsaturated Vegetable Oils. Industrial & Engineering Chemistry Research (2013). However, the effect of other structural factors, such as trans- and cis-configurations, positional isomers, terminal and internal branches, etc., on the physical properties has not yet been clarified. As such, the present effort reports on structure-controlled dimers and quatrimers and the effect of saturation, molecular size and positional isomerism on their physical properties.

### BRIEF DESCRIPTION OF THE DRAWINGS

FIG. 1 depicts a ¹H-NMR spectrum of D2, representative of the synthesized oligomers.

FIG. 2 depicts a ¹³C-NMR spectrum of D2, representative of the synthesized dimers and quatrimers.

FIG. 3a depicts DTG curves of dimers and quatrimers.

FIG. 3b depicts the onset temperature of degradation (T_{On}^{d},) of dimers and quatrimers determined at the intersection of the baseline (0% weight loss line) and the tangent at the first inflexion point.

FIG. 4a depicts DSC cooling profiles of dimers and quatrimers.

FIG. 4b depicts onset temperatures of crystallization of dimers and quatrimers.

FIG. 4c depicts offset temperatures of crystallization of dimers and quatrimers.

FIG. 4d depicts the enthalpy of crystallization (ΔH_{C}) of dimers and quatrimers.

FIG. 4e depicts the peak temperature of the first exotherm in the cooling thermograms of dimers and quatrimers.

FIG. 4f depicts the peak temperature of the second exotherms in the cooling thermograms of dimers and quatrimers.

FIG. 5a depicts the DSC heating profiles of dimers and quatrimers.

FIG. 5b depicts the offset temperature of melting of dimers and quatrimers.

FIG. 5c depicts the peak temperature of the last two endotherms of the dimers and quatrimers.

### DETAILED DESCRIPTION

The present application relates to triacylglycerol oligomers derived from the metathesis of natural oils. These oligomers are structure controlled dimers and quatrimers, and the effect of saturation, molecular size, and positional isomerization are also described.

A series of series of model dimers and quatrimers with controlled structures were synthesized from 1,3-substituted glycerol; 1,18-octadec-9-enedioic acid and 2,3-dihydroxypropyl oleate, and their structures were characterized by ¹H-NMR and ¹³C-NMR. Additionally for the model dimers and quatrimers, the thermal stability, crystallization and melting behavior were investigated as a function of saturation, isomerism and molecular mass, using TGA and DSC.

The materials used to synthesize such oligomers were are follows: stearoyl chloride (98%), oleoyl chloride (85%), oleic acid (90%), 1,3-dihydroxyacetone (99%), glycerol (99%), solketal (98%), pyridine (99%), N,N′-dicyclohexylcarbodiimide (DCC), 4-dimethylaminopyridine (DMAP), Grubbs 2^{nd} generation metathesis catalyst, and sodium borohydride were purchased from Sigma-Aldrich. 1,18-octadec-9-enedioic acid and 1-substituted-2,3-dihydroxypropane were prepared in our laboratories. Their synthesis and characterization were reported by Li, S., L. Bouzidi, and S. S. Narine, Synthesis and Physical Properties of Triacylglycerol Oligomers: Examining the Physical Functionality Potential of Self-Metathesized Highly Unsaturated Vegetable Oils. Industrial & Engineering Chemistry Research (2013). Chloroform was purified by distillation over calcium hydride.

The structures of the dimers and quatrimers of the present application are shown in Scheme 1 below:

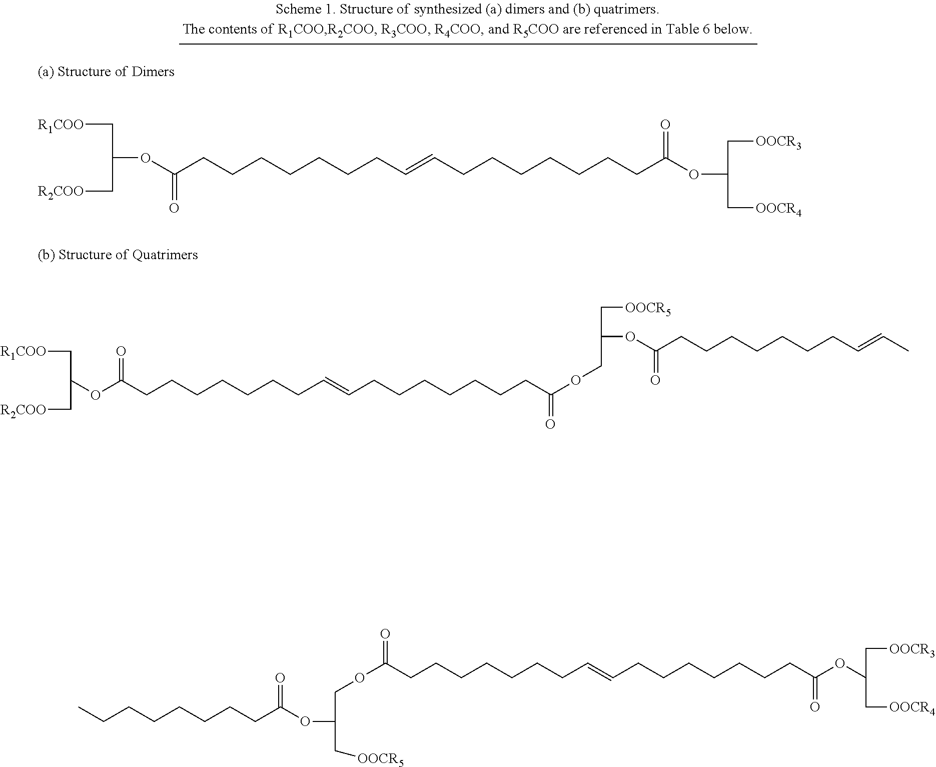

The dimers and quatrimers were prepared at room temperature (often between 17-27° C.), and for a time period overnight (often between 8-16 hours), by reacting a fatty carboxylic acid (or its acid halide, such as an acid chloride created by reacting a fatty carboxylic acid with a chlorinating agent, such as thionyl chloride, phosphorus trichloride, oxalylchloride or phosphorus pentachloride) and a fatty alcohol with a condensing agent and a catalyst. Additionally, the dimer and quatrimers may be prepared via a metathesis route.

Metathesis (either self-metathesis or cross-metathesis) is a catalytic reaction that involves the interchange of alkylidene units among compounds containing one or more double bonds (i.e., olefinic compounds) via the formation and cleavage of the carbon-carbon double bonds. The metathesis catalyst in this reaction may include any catalyst or catalyst system that catalyzes a metathesis reaction. Suitable homogeneous metathesis catalysts include combinations of a transition metal halide or oxo-halide (e.g., WOCl₄ or Wok) with an alkylating cocatalyst (e.g., Me₄Sn). Homogeneous catalysts may be well-defined alkylidene (or carbene) complexes of transition metals, particularly Ru, Mo, or W. These include first and second-generation Grubbs catalysts, Grubbs-Hoveyda catalysts, and the like. Suitable alkylidene catalysts have the general structure:

M[X¹X²L¹L²(L³)ₙ]=Cₘ═C(R¹)R²

where M is a Group 8 transition metal, L¹, L², and L³ are neutral electron donor ligands, n is 0 (such that L³ may not be present) or 1, m is 0, 1, or 2, X¹ and X² are anionic ligands, and R¹ and R² are independently selected from H, hydrocarbyl, substituted hydrocarbyl, heteroatom-containing hydrocarbyl, substituted heteroatom-containing hydrocarbyl, and functional groups. Any two or more of X¹, X², L¹, L², L³, R¹ and R² may form a cyclic group and any one of those groups may be attached to a support.

First-generation Grubbs catalysts fall into this category where m=n=0 and particular selections are made for n, X¹, X², L¹, L², L³, R¹ and R² as described in U.S. Pat. Appl. Publ. No. 2010/0145086 (“the '086 publication”), the teachings of which related to all metathesis catalysts are incorporated herein by reference. Second-generation Grubbs catalysts also have the formula described above, but L¹ is a carbene ligand where the carbene carbon is flanked by N, O, S, or P atoms, (e.g., by two N atoms). The carbene ligand may be part of a cyclic group. Examples of suitable second-generation Grubbs catalysts also appear in the '086 publication.

In another class of suitable alkylidene catalysts, L¹ is a strongly coordinating neutral electron donor as in first- and second-generation Grubbs catalysts, and L² and L³ are weakly coordinating neutral electron donor ligands in the form of optionally substituted heterocyclic groups. Thus, L² and L³ are pyridine, pyrimidine, pyrrole, quinoline, thiophene, or the like. In yet another class of suitable alkylidene catalysts, a pair of substituents is used to form a bi- or tridentate ligand, such as a biphosphine, dialkoxide, or alkyldiketonate. Grubbs-Hoveyda catalysts are a subset of this type of catalyst in which L² and R² are linked. A neutral oxygen or nitrogen may coordinate to the metal while also being bonded to a carbon that is α-, β-, or γ-with respect to the carbene carbon to provide the bidentate ligand. Examples of suitable Grubbs-Hoveyda catalysts appear in the '086 publication.

The structures below (Scheme 2) provide just a few illustrations of suitable catalysts that may be used:

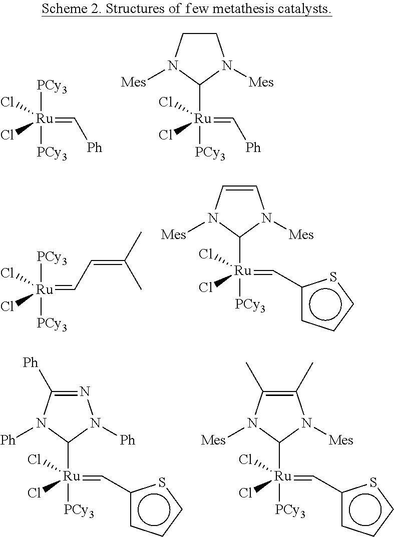

Heterogeneous catalysts suitable for use in the self- or cross-metathesis reactions include certain rhenium and molybdenum compounds as described, e.g., by J. C. Mol in Green Chem. 4 (2002) 5 at pp. 11-12. Particular examples are catalyst systems that include Re207 on alumina promoted by an alkylating cocatalyst such as a tetraalkyl tin lead, germanium, or silicon compound. Others include MoCl₃ or MoCl₅ on silica activated by tetraalkyltins. For additional examples of suitable catalysts for self- or cross-metathesis, see U.S. Pat. No. 4,545,941, the teachings of which are incorporated herein by reference, and references cited therein. See also J. Org. Chem. 46 (1981) 1821; J. Catal. 30 (1973) 118; Appl. Catal. 70 (1991) 295; Organometallics 13 (1994) 635; Olefin Metathesis and Metathesis Polymerization by Ivin and Mol (1997), and Chem. & Eng. News 80(51), Dec. 23, 2002, p. 29, which also disclose useful metathesis catalysts. Illustrative examples of suitable catalysts include ruthenium and osmium carbene catalysts as disclosed in U.S. Pat. Nos. 5,312,940, 5,342,909, 5,710,298, 5,728,785, 5,728,917, 5,750,815, 5,831,108, 5,922,863, 6,306,988, 6,414,097, 6,696,597, 6,794,534, 7,102,047, 7,378,528, and U.S. Pat. Appl. Publ. No. 2009/0264672 A1, and PCT/US2008/009635, pp. 18-47, all of which are incorporated herein by reference. A number of metathesis catalysts that may be advantageously employed in metathesis reactions are manufactured and sold by Materia, Inc. (Pasadena, Calif.).

The condensing agent used in the dimer and quatrimer synthesis often is a carbodiimide, represented by the formula: R¹N═C═NR² wherein R¹ and R² are alkyl groups containing from 1 to 18 carbon atoms, cycloalkyl groups containing 5 to 10 carbon atoms and aryl groups, which term includes alkaryl and arylalkyl groups, containing 5 to 18 carbon atoms. Non-limiting examples of such carbodiimides are dimethyl carbodiimide, diisopropyl carbodiimide, diisobutyl carbodiimide, dioctyl carbodiimide, tert-butyl isopropyl carbodiimide, dodecyl isopropyl carbodiimide, dicylohexyl carbodiimide, diphenyl carbodiimide, di-o-tolyl carbodiimide, bis(2,6-diethylphenyl) carbodiimide, bis(2,6-diisopropylphenyl carbodiimide, di-beta-naphthyl carbodiimide, benzyl isoopropyl carbodiimide, phenyl-o-tolyl carbodiimide, and dicyclohexylcarbodiimide (DCC).

The catalyst may include a base, with non-limiting examples such as a triethyl amine, tripropyl amine, tributyl amine, pyridine and 4-dimethylamino pyridine or other pyridine derivative, and 4-dimethylaminopyridine (DMAP).

The solvent used in the synthesis may be chosen from the group including but not limited to aliphatic hydrocarbons (e.g., hexane and cyclohexane), organic esters (i.e. ethyl acetate), aromatic hydrocarbons (e.g., benzene and toluene), ethers (e.g., dioxane, tetrahydrofuran, ethyl ether, tert-butyl methyl ether), halogenated hydrocarbons (e.g., methylene chloride and chloroform).

The fatty carboxylic acid is derived from a natural oil, with non-limiting examples such as canola oil, rapeseed oil, coconut oil, corn oil, cottonseed oil, olive oil, palm oil, peanut oil, safflower seed oil, sesame seed oil, soybean oil, sunflower oil, linseed oil, palm kernel oil, tung oil, jojoba oil, jatropha oil, mustard oil, camelina oil, pennycress oil, hemp oil, algal oil, castor oil, lard, tallow, poultry fat, yellow grease, fish oil, tall oils, and mixtures thereof. Optionally, the natural oil may be partially and/or fully hydrogenated, and may also be refined, bleached, and/or deodorized.

Natural oils may include triacylglycerols (TAGs) of saturated and unsaturated fatty acids. Suitable fatty acids may be saturated or unsaturated (monounsaturated or polyunsaturated) fatty acids, and may have carbon chain lengths of 3 to 36 carbon atoms. Such saturated or unsaturated fatty acids may be aliphatic, aromatic, saturated, unsaturated, straight chain or branched, substituted or unsubstituted, fatty acids, and mono-, di-, tri-, and/or poly-acid variants, hydroxy-substituted variants, aliphatic, cyclic, alicyclic, aromatic, branched, aliphatic- and alicyclic-substituted aromatic, aromatic-substituted aliphatic and alicyclic groups, and heteroatom substituted variants thereof. Any unsaturation may be present at any suitable isomer position along the carbon chain to a person skilled in the art.

Some non-limiting examples of saturated fatty acids include propionic, butyric, valeric, caproic, enanthic, caprylic, pelargonic, capric, undecylic, lauric, tridecylic, myristic, pentadecanoic, palmitic, margaric, stearic, nonadecyclic, arachidic, heneicosylic, behenic, tricosylic, lignoceric, pentacoyslic, cerotic, heptacosylic, carboceric, montanic, nonacosylic, melissic, lacceroic, psyllic, geddic, and ceroplastic acids.

Some non-limiting examples of unsaturated fatty acids include butenoic, pentenoic, hexenoic, pentenoic, octenoic, nonenoic acid, decenoic acid, undecenoic acid, dodecenoic acid, tridecenoic, tetradecenoic, pentadecenoic, palmitoleic, palmitelaidic oleic, ricinoleic, vaccenic, linoleic, linolenic, elaidic, eicosapentaenoic, behenic and erucic acids. Some unsaturated fatty acids may be monounsaturated, diunsaturated, triunsaturated, tetraunsaturated or otherwise polyunsaturated, including any omega unsaturated fatty acids.

In a triacylglycerol, each of the carbons in the triacylglycerol molecule may be numbered using the stereospecific numbering (sn) system. Thus one fatty acyl chain group is attached to the first carbon (the sn-1 position), another fatty acyl chain is attached to the second, or middle carbon (the sn-2 position), and the final fatty acyl chain is attached to the third carbon (the sn-3 position). The triacylglycerols described herein may include saturated and/or unsaturated fatty acids present at the sn-1, sn-2, and/or sn-3 position.

The alcohol used in the synthesis is often a fatty alcohol of between 2 and 30 carbon atoms. The fatty alcohols include monohydric and polyhydric fatty alcohols, particularly those containing 2 to 30 carbon atoms exhibiting straight-chain or branched-chain structure, which are saturated or unsaturated (containing one or more carbon-carbon double bonds). Non-limiting examples of representative alcohols include oleic, linoleic, linolenic, lauric, caproic, erucic, myristic and palmitic alcohols, as well as mixtures of any of the foregoing alcohols. In some embodiments, the alcohol may be 2,3-dihydroxypropyl-3-oleyl glycerol.

The dimers and quatrimers were prepared following the synthesis procedure shown in Scheme 3. The compounds were characterized by NMR and/or MS. The NMR and MS data are referenced later in this document.

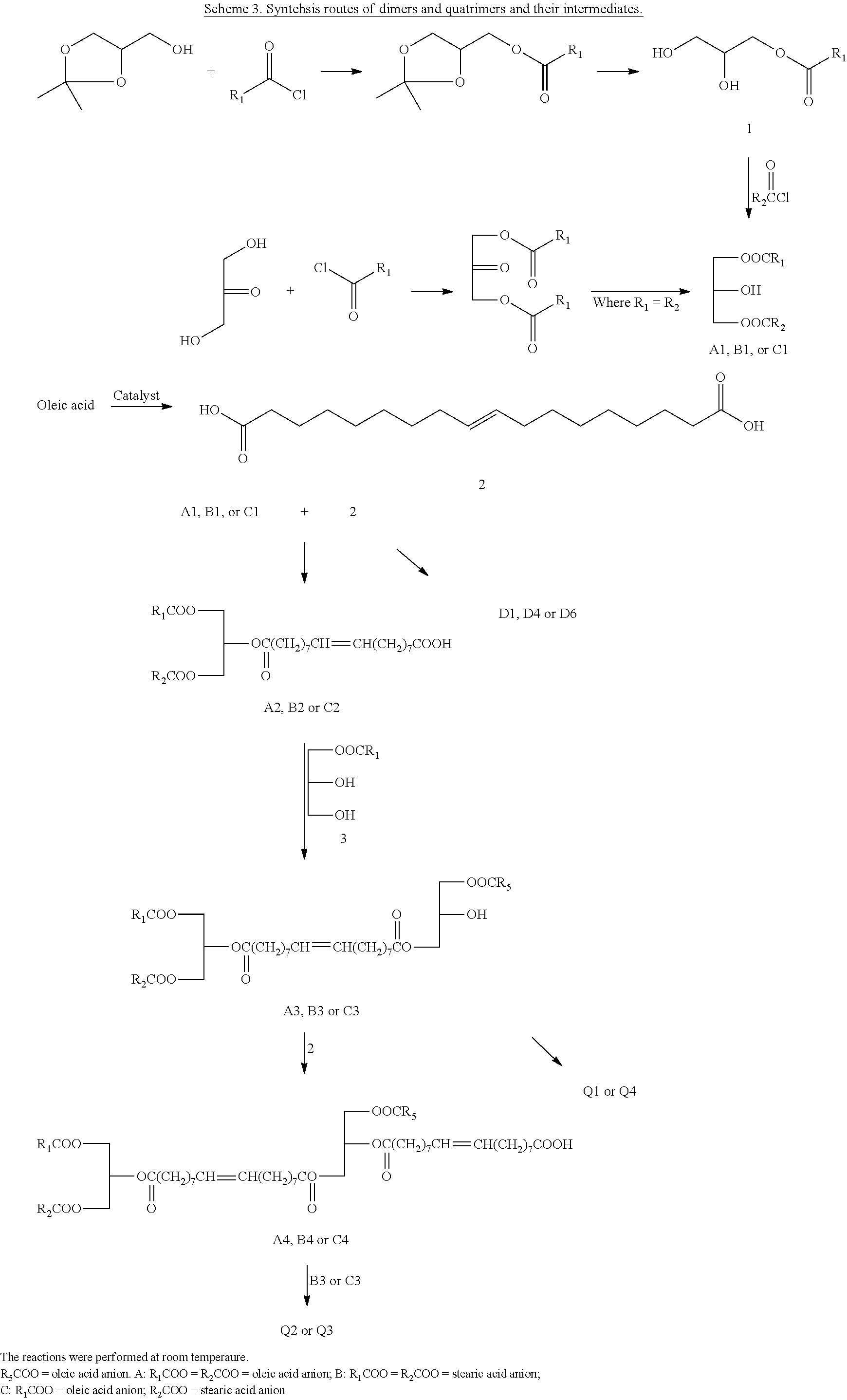

The dimers (D1-D6) and quatrimers (Q1-Q4), with representative structures and systematic names referenced below in Table 2 were synthesized from 1,3-substituted glycerol (A1, B1, and C1, with representative structures and systematic names referenced below in Table 2), 1,18-octadec-9-enedioic acid (2, in Scheme 3) and 2,3-dihydroxypropyl oleate (1, in Scheme 3) by Steglich esterification. 4-dimethylaminopyridine (DMAP) was used as catalyst and N,N′-dicyclohexylcarbodiimide (DCC) as the condensing agent. The specific intermediates used to prepare each dimer and quatrimer are listed in Table 1 below.

TABLE 1
Intermediates used to prepare the dimers and
quatrimers. The nomenclature used refer to
the compounds are labeled in Scheme 3.

| Oligomers (dimers, D1-D6, | |
|---|---|
| and quatrimers, Q1-Q4) | Obtained From |
| D1 | A1 + 2 |
| D2 | A1 + B2 |
| D3 | A1 + C1 |
| D4 | B1 + 2 |
| D5 | B2 + C1 |
| D6 | C1 + 2 |
| Q1 | A3 + 2 |
| Q2 | A4 + B3 |
| Q3 | A4 + C3 |
| Q4 | B3 + 2 |

1,3-substituted glycerol was synthesized from 1,3-disubstituted glyceroloxypropan-2-one, prepared from 1,3-dihydroxylacetone and oleic or stearic acid or their chlorides, following known procedures. 1,18-octadec-9-enedioic acid was produced by self-metathesis of oleic acid using Grubbs 2^{nd} generation metathesis catalyst. The trans-nature of the double bond on the alkyl chain of the diacid of this compound has been confirmed in a previous study that used the same synthesis procedure.

1,3-disubstituted-2-hydroxypropane (A1, B1 or C1) was synthesized following known procedures. An intermediate, 1,3-disubstituted-2-oxopropane was prepared from 1,3-dihydroxylacetone and fatty acid (or chloride) with DMAP as catalyst and DCC as the condensing agent (or in the presence of pyridine). The resultant ketone was reduced by NaBH₄ in a solution of THF.

1,2-Isopropylidene-3-substituted glycerol was synthesized by esterification of solketal and fatty acid (or chloride). 1-substituted-2,3-dihydroxypropane (1, in Scheme 1) was prepared by deprotecting 1,2-Isopropylidene-3-substituted glycerol with concentrated HCl in dioxane.

The mono-acids (A2, B2 or C2, with representative structures and systematic names referenced below in Table 2) were prepared separately from 1,3-disubstituted-2-hydroxypropane (A1, B1 or C1) and 1,18-octadec-9-enedioic acid by controlling their ratios.

The mono-ols with sn-2 OH (A3, B3 or C3 in Scheme 3, with representative structures and systematic names referenced below in Table 2) were prepared from mono-acids (A2, B2 or C2) and 1-substituted-2,3-dihydroxypropane by controlling their ratio. The by-product (15%) with sn-1 OH (A3-II, B3-II or 03-II) was carefully removed from the mono-ols with column chromatography. All the reactions were carried out at room temperature to avoid the conversion of cis-geometry into trans-geometry, a phenomenon that is known to occur at a high temperature. All the synthesized compounds, including the intermediates, were carefully purified to provide that the targeted structures (shown in Scheme 1) were obtained. The oligomers (dimers and quatrimers) were classified into symmetric and asymmetric structures depending on the nature of their terminal chains. The oligomers that have the same neighboring fatty acid chains, such as dimers D1, D3, D5 and D6, were taken as symmetric structures and the oligomers with mixed neighboring fatty acid chains, such as dimers D2 and D4, were taken as asymmetric structures.

The representative systematic names and structures of the dimers, quatrimers and their intermediates are shown in Table 2 below:

TABLE 2
Representative Structures of A1, B1, and C1, and A2, B2, and C2, and A3, B3, and C3
A1: 2-hydroxypropane-1,3-diyl dioleate

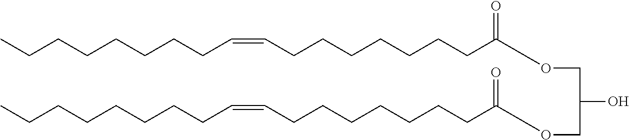 [SMILES: CCCCCCCC/C=C\CCCCCCCC(=O)OCC(O)COC(=O)CCCCCCC/C=C\CCCCCCCC]

B1: 2-hydroxypropane-1,3-diyl distearate

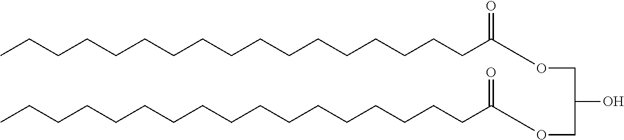 [SMILES: CCCCCCCCCCCCCCCCCC(=O)OCC(O)COC(=O)CCCCCCCCCCCCCCCCC]

C1: 2-hydroxy-3-(stearoyloxy)propyl oleate

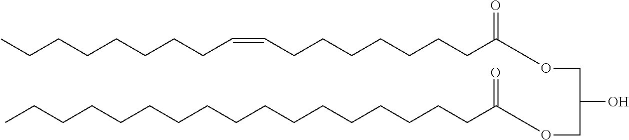 [SMILES: CCCCCCCC/C=C\CCCCCCCC(=O)OCC(O)COC(=O)CCCCCCCCCCCCCCCCC]

A2: 18-((1,3-bis(oleoyloxy)propan-2-yl)oxy)-18-oxooctadec-9-enoic acid

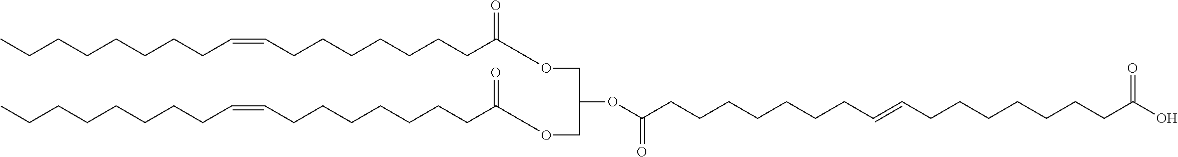 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)O]

B2: 18-((1,3-bis(stearoyloxy)propan-2-yl)oxy)-18-oxooctadec-9-enoic acid

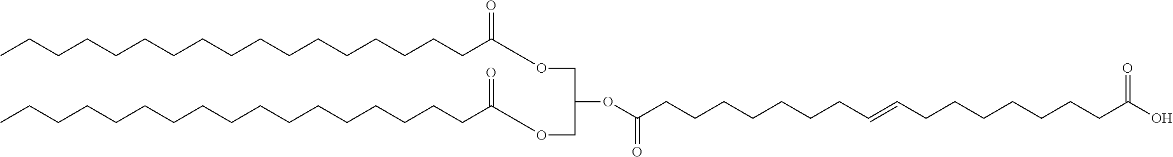 [SMILES: CCCCCCCCCCCCCCCCCC(=O)OCC(COC(=O)CCCCCCCCCCCCCCCCC)OC(=O)CCCCCCC/C=C/CCCCCCCC(=O)O]

C2: 18-((1-(oleoyloxy)-3-(stearoyloxy)propan-2-yl)oxy)-18-oxooctadec-9-enoic acid

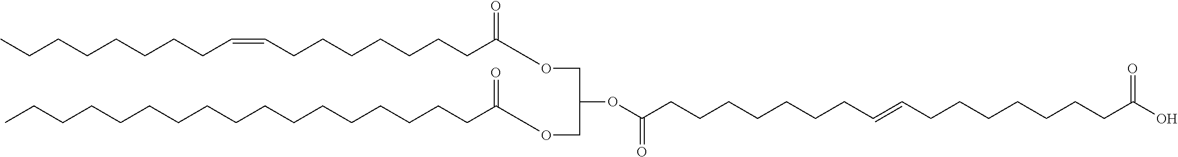 [SMILES: CCCCCCCC/C=C\CCCCCCCC(=O)OCC(COC(=O)CCCCCCCCCCCCCCCCC)OC(=O)CCCCCCC/C=C/CCCCCCCC(=O)O]

Representative Structures of A3, B3, and C3, and A4, B4, and C4
A3: 1-(1,3-bis(oleoyloxy)propan-2-yl)18-(2-hydroxy-3-(oleoyloxy)propyl)octadec-9-enedioate

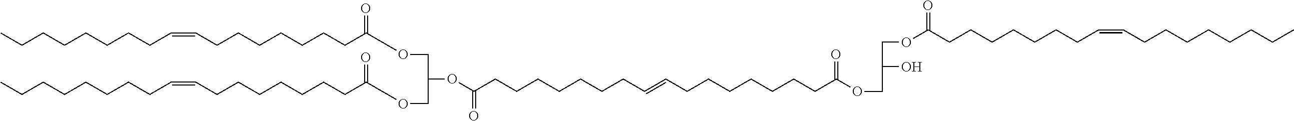 [SMILES: CCCCCCCC/C=C\CCCCCCCC(=O)OCC(O)COC(=O)CCCCCCC/C=C/CCCCCCCC(=O)OC(COC(=O)CCCCCCC/C=C\CCCCCCCC)COC(=O)CCCCCCC/C=C\CCCCCCCC]

B3: 1-(1,3-bis(stearoyloxy)propan-2-yl)18-(2-hydroxy-3-(oleoyloxy)propyl)octadec-9-enedioate

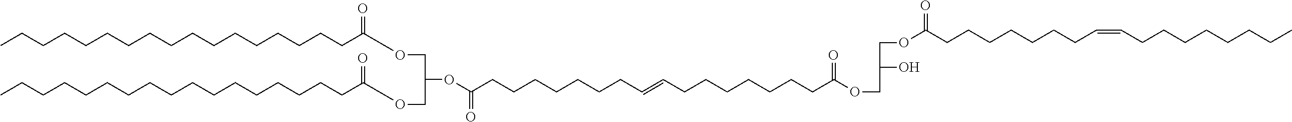 [SMILES: CCCCCCCC/C=C\CCCCCCCC(=O)OCC(O)COC(=O)CCCCCCC/C=C/CCCCCCCC(=O)OC(COC(=O)CCCCCCCCCCCCCCCCC)COC(=O)CCCCCCCCCCCCCCCCC]

C3: 1-(2-hydroxy-3-(oleoyloxy)propyl)18-(1-(oleoyloxy)-3-(stearoyloxy)propan-2-yl)octadec-9-enedioate

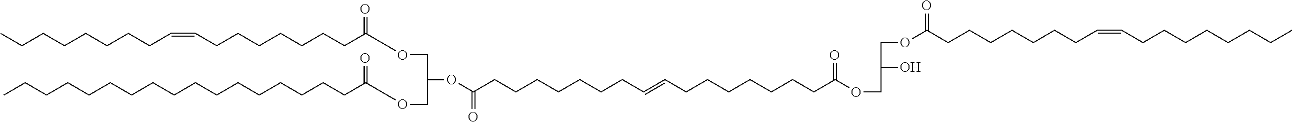 [SMILES: CCCCCCCC/C=C\CCCCCCCC(=O)OCC(O)COC(=O)CCCCCCC/C=C/CCCCCCCC(=O)OC(COC(=O)CCCCCCC/C=C\CCCCCCCC)COC(=O)CCCCCCCCCCCCCCCCC]

A4: 20,42-bis((oleoyloxy)methyl)-18,23,40,45-tetraoxo-19,22,41,44-tetraoxadohexaconta-9,31,53-trien-1-oic acid

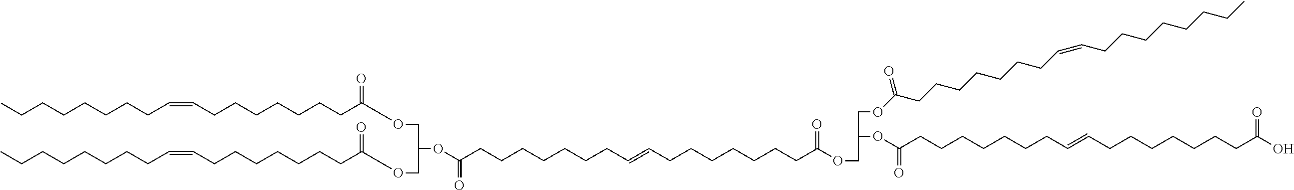 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OCC(COC(=O)CCCCCCC/C=C\CCCCCCCC)OC(=O)CCCCCCC/C=C/CCCCCCCC(=O)O]

B4: 20-((oleoyloxy)methyl)-18,23,40,45-tetraoxo-42-((stearoyloxy)methyl)-19,22,41,44-tetraoxadohexaconta-9,31-dien-1-oic acid

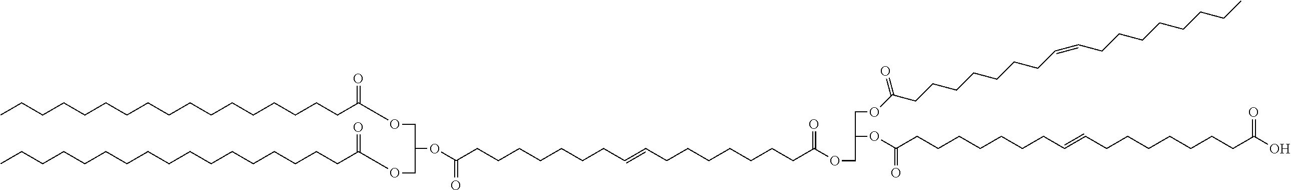 [SMILES: CCCCCCCC/C=C\CCCCCCCC(=O)OCC(COC(=O)CCCCCCC/C=C/CCCCCCCC(=O)OC(COC(=O)CCCCCCCCCCCCCCCCC)COC(=O)CCCCCCCCCCCCCCCCC)OC(=O)CCCCCCC/C=C/CCCCCCCC(=O)O]

C4: 20-((oleoyloxy)methyl)-18,23,40,45-tetraoxo-42-((stearoyloxy)methyl)-19,22,41,44-tetraoxadohexaconta-9,31,53-trien-1-oic acid

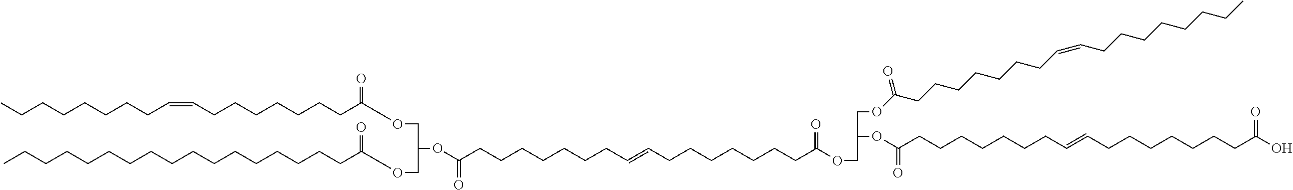 [SMILES: CCCCCCCC/C=C\CCCCCCCC(=O)OCC(COC(=O)CCCCCCC/C=C/CCCCCCCC(=O)OC(COC(=O)CCCCCCC/C=C\CCCCCCCC)COC(=O)CCCCCCCCCCCCCCCCC)OC(=O)CCCCCCC/C=C/CCCCCCCC(=O)O]

Representative Structures of Dimers D1, D2, D3, D4, D5, and D6
D1: bis(1,3-bis(oleoyloxy)propan-2-yl)octadec-9-enedioate

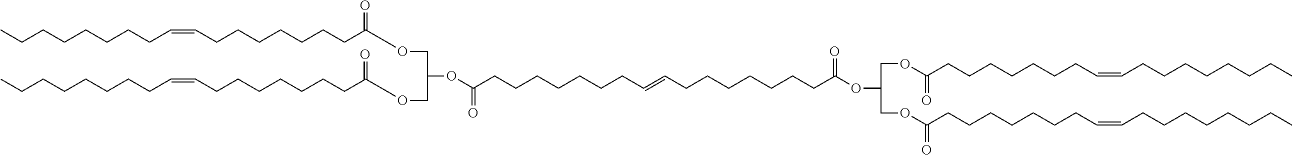 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OC(COC(=O)CCCCCCC/C=C\CCCCCCCC)COC(=O)CCCCCCC/C=C\CCCCCCCC]

D2: 1-(1,3-bis(oleoyloxy)propan-2-yl)18-(1-(oleoyloxy)-3-(stearoyloxy)propan-2-yl)octadec-9-enedioate

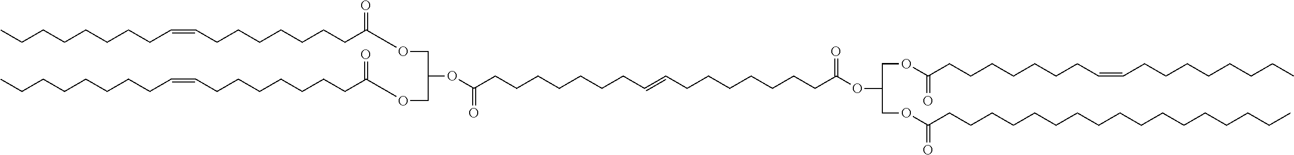 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OC(COC(=O)CCCCCCC/C=C\CCCCCCCC)COC(=O)CCCCCCCCCCCCCCCCC]

D3: 1-(1,3-bis(oleoyloxy)propan-2-yl)18-(1,3-bis(stearoyloxy)propan-2-yl)octadec-9-enedioate

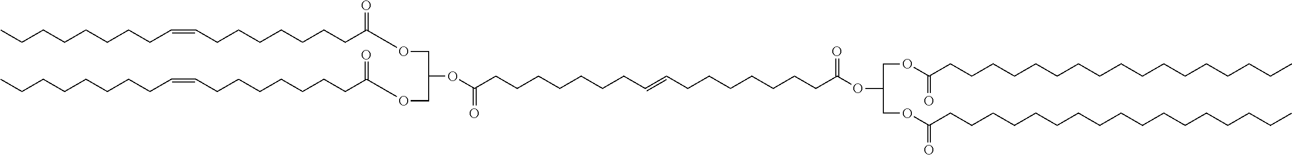 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OC(COC(=O)CCCCCCCCCCCCCCCCC)COC(=O)CCCCCCCCCCCCCCCCC]

D4: 1-(1-(oleoyloxy)-3-(stearoyloxy)propan-2-yl)18-(1-(oleoyloxy)-3-(stearoyloxy)propan-2-yl)octadec-9-enedioate

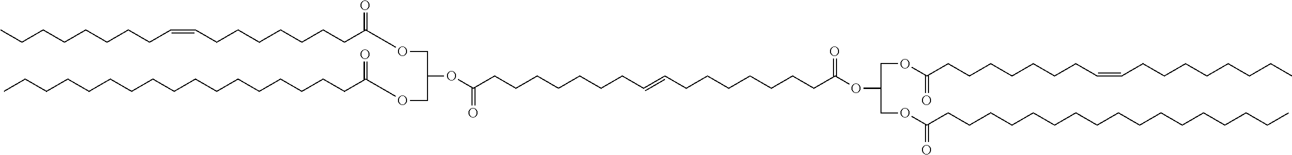 [SMILES: CCCCCCCC/C=C\CCCCCCCC(=O)OCC(COC(=O)CCCCCCCCCCCCCCCCC)OC(=O)CCCCCCC/C=C/CCCCCCCC(=O)OC(COC(=O)CCCCCCC/C=C\CCCCCCCC)COC(=O)CCCCCCCCCCCCCCCCC]

D5: 1-(1,3-bis(stearoyloxy)propan-2-yl)18-(1-(oleoyloxy)-3-(stearoyloxy)propan-2-yl)octadec-9-enedioate

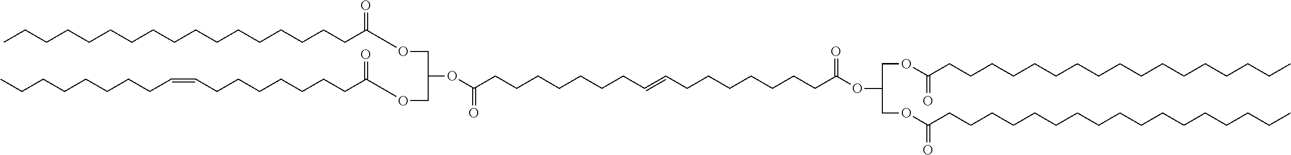 [SMILES: CCCCCCCC/C=C\CCCCCCCC(=O)OCC(COC(=O)CCCCCCCCCCCCCCCCC)OC(=O)CCCCCCC/C=C/CCCCCCCC(=O)OC(COC(=O)CCCCCCCCCCCCCCCCC)COC(=O)CCCCCCCCCCCCCCCCC]

D6: bis(1,3-bis(stearoyloxy)propan-2-yl)octadec-9-enedioate

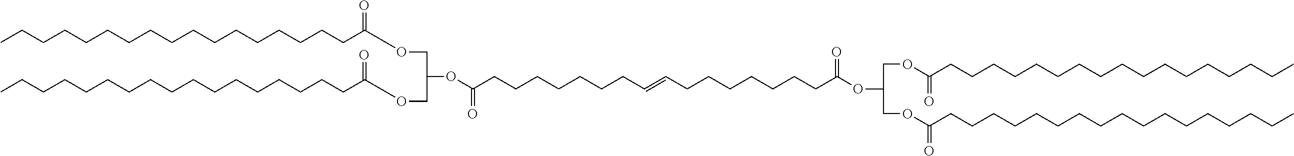 [SMILES: CCCCCCCCCCCCCCCCCC(=O)OCC(COC(=O)CCCCCCCCCCCCCCCCC)OC(=O)CCCCCCC/C=C/CCCCCCCC(=O)OC(COC(=O)CCCCCCCCCCCCCCCCC)COC(=O)CCCCCCCCCCCCCCCCC]

Representative Structures of Quatrimers Q1, Q2, Q3, and Q4
Q1: bis(1,3-bis(oleoyloxy)propan-2-yl)O′¹,O¹-(((octadec-9-enedioyl)bis(oxy))bis(3-(oleoyloxy)propane-2,1-diyl))bis(octadec-9-enedioate)

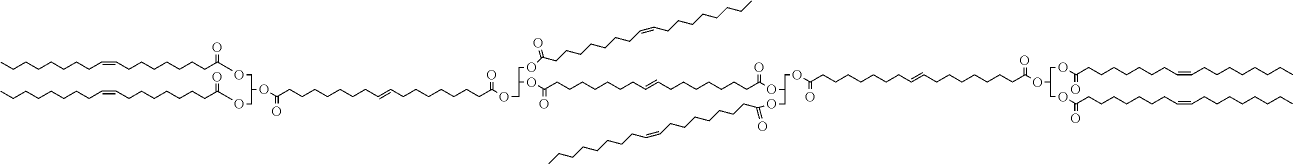 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OCC(COC(=O)CCCCCCC/C=C\CCCCCCCC)OC(=O)CCCCCCC/C=C/CCCCCCCC(=O)OC(COC(=O)CCCCCCC/C=C\CCCCCCCC)COC(=O)CCCCCCC/C=C/CCCCCCCC(=O)OC(COC(=O)CCCCCCC/C=C\CCCCCCCC)COC(=O)CCCCCCC/C=C\CCCCCCCC]

Q2: 1-(2,23-bis((oleoyloxy)methyl)-4,21,26,43,48-pentaoxo-45-((stearoyloxy)methyl)-3,22,25,44,47-pentaoxapentahexaconta-12,34,56-trien-1-yl)18-(1,3-bis(oleoyloxy)propan-2-yl)octadec-9-enedioate

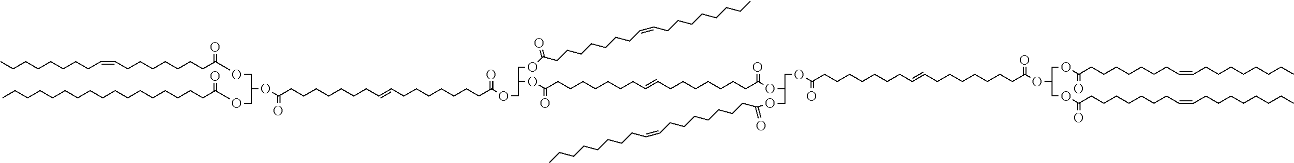 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OCC(COC(=O)CCCCCCC/C=C\CCCCCCCC)OC(=O)CCCCCCC/C=C/CCCCCCCC(=O)OC(COC(=O)CCCCCCC/C=C\CCCCCCCC)COC(=O)CCCCCCC/C=C/CCCCCCCC(=O)OC(COC(=O)CCCCCCC/C=C\CCCCCCCC)COC(=O)CCCCCCCCCCCCCCCCC]

Q3: 1-(2,23-bis((oleoyloxy)methyl)-4,21,26,43,48-pentaoxo-45-((stearoyloxy)methyl)-3,22,25,44,47-pentaoxapentahexaconta-12,34-dien-1-yl)18-(1,3-bis(oleoyloxy)propan-2-yl)octadec-9-enedioate

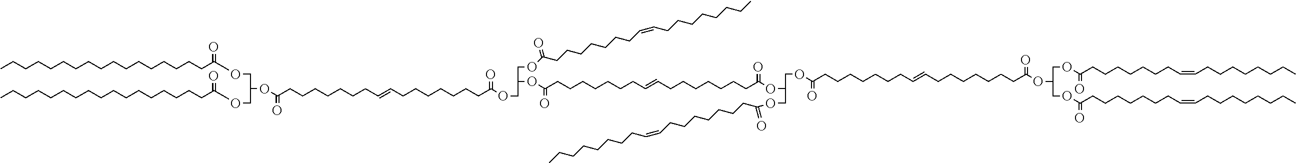 [SMILES: CCCCCCCC/C=C\CCCCCCCC(=O)OCC(COC(=O)CCCCCCC/C=C\CCCCCCCC)OC(=O)CCCCCCC/C=C/CCCCCCCC(=O)OCC(COC(=O)CCCCCCC/C=C\CCCCCCCC)OC(=O)CCCCCCC/C=C/CCCCCCCC(=O)OC(COC(=O)CCCCCCC/C=C\CCCCCCCC)COC(=O)CCCCCCC/C=C/CCCCCCCC(=O)OC(COC(=O)CCCCCCCCCCCCCCCCC)COC(=O)CCCCCCCCCCCCCCCCC]

Q4: bis(1-(oleoyloxy)-3-(stearoyloxy)propan-2-yl)O′¹,O¹-(((octadec-9-enedioyl)bis(oxy))bis(3-(oleoyloxy)propane-2,1-diyl))bis(octadec-9-enedioate)

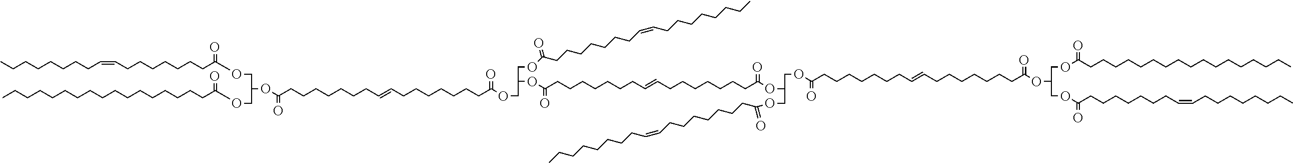 [SMILES: CCCCCCCC/C=C\CCCCCCCC(=O)OCC(COC(=O)CCCCCCC/C=C/CCCCCCCC(=O)OC(COC(=O)CCCCCCC/C=C\CCCCCCCC)COC(=O)CCCCCCCCCCCCCCCCC)OC(=O)CCCCCCC/C=C/CCCCCCCC(=O)OC(COC(=O)CCCCCCC/C=C\CCCCCCCC)COC(=O)CCCCCCC/C=C/CCCCCCCC(=O)OC(COC(=O)CCCCCCC/C=C\CCCCCCCC)COC(=O)CCCCCCCCCCCCCCCCC]

### Synthesis of 2,3-dihydroxypropyl Compounds

### 1,2-isopropylidene-3-substituted glycerol (1)

Fatty acid chloride (100 mmol) in (100 mL) chloroform was slowly added to a solution of (100 mmol) solketal in (200 mL) chloroform and (150 mmol) pyridine. The reaction mixture was stirred for 2 days at room temperature. The chloroform solution was washed sequentially with water, 5% HCl, water, 4% NaHCO₃, and brine, then dried on Na₂SO₄. After removing the solvent, the resultant mixture was used in the next step without further separation.

### 2,3-dihydroxypropyl Compounds

Concentrated HCl (0.2 mol) was added to 1,2-isopropylidene-3-glycerol (100 mmol) in 500 ml dioxane. The reaction was stirred at room temperature for 5 hours.

The mixture was then diluted by water and extracted with ethyl acetate. The ethyl acetate layer was washed sequentially with 4% NaHCO₃ and water, and dried on Na₂SO₄. The organic solvent was removed and the residue was separated by column chromatography with ethyl acetate/hexanes=1:4 to 1:1.

### Synthesis of 1,3-disubstituted Glycerol (A1, B1 or C1)

### Synthesis of 1,3-disubstituted glyceroloxypropan-2-one

Chloride (82.53 mmol) was added to a solution of (41.27 mmol) 1,3-dihydroxylacetone in (160 mL) chloroform, followed by the dropwise addition of (90.79 mmol) pyridine. The reaction mixture was stirred at room temperature overnight. The reaction mixture was then diluted with 160 mL chloroform. The organic layer was washed with water (3×300 mL), followed sequentially by 5% HCl (2×300 mL), water (2×300 mL), 4% NaHCO₃ (2×300 mL), and water (3×300 mL). The organic layer was dried on Na₂SO₄. After chloroform was removed, the residue was recrystallized from 2-propanol or purified by column chromatography.

### Synthesis of 1,3-substituted glycerol

NaBH₄ (51.86 mmol) in water (a small quantity) was slowly added to a solution of (34.57 mmol) 1,3-diglyceroloxypropane-2-one in 300 ml THF at 5° C. The reaction mixture was stirred at 5° C. for 30 min and quenched by 5% HCl. 300 ml water was added and the mixture was extracted with 400 ml chloroform. The organic layer was washed sequentially with water (3×400 mL), 4% NaHCO₃ (2×300 mL) and water (3×400 mL). The organic layer was dried on Na₂SO₄. After chloroform was removed, the residue was recrystallized from hexane or purified by column chromatography.

### Synthesis of 1,18-Octadec-9-enedioic acid (2)

Oleic acid (76 g) was transferred into a 250 mL three-necked round bottom flask and stirred at 45° C. under nitrogen gas for 0.5 hours. Grubbs 2^{nd} generation catalyst (85 mg) was added. The reaction mixture was stirred at 45° C. for around 5 min. The diacid began to precipitate from the reaction mixture. The reaction was kept at this temperature for 24 hours, and then quenched with ethyl vinyl ether (15 mL). The excess ether was removed under reduced pressure. The residue was purified by recrystallization from ethyl acetate and hexanes (1:2) to give 29.75 g of product as a white solid.

Route for the Synthesis of Dimers, Quatrimers and their Related Intermediates

The dimers, quatrimers and their related intermediates were prepared using the formulations (acid, alcohol, and catalyst amounts) listed in Table 3 below. The synthetic route was as follows: To a solution of alcohol and acid in 10 mL CHCl₃ was added 0.2 mmol DMAP under the protection of N₂, followed by 1.2 mmol DCC. The reaction was carried out at room temperature overnight. The precipitated dicyclohexylurea was removed by filtration. The organic phase was diluted with 10 mL chloroform then washed sequentially with water (3×20 mL), 4% aqueous NaHCO₃ (2×200 mL) and brine (3×200 mL), and then dried over Na₂SO₄. After filtration, the filtrate was concentrated with a rotary evaporator and the residue was purified by column chromatography with ethyl acetate and hexanes as the eluent.

TABLE 3
Stoichiometry of synthesizing dinners, quatrimers
and their related intermediates.

| | Alcohol | | Acid | | | |
|---|---|---|---|---|---|---|
| | | Amount | | Amount | DMAP | DCC |
| Compound | SM | (mmol) | SM | (mmol) | (mmol) | (mmol) |
| A2 | A1 | 1 | 2 | 1.2 | 0.2 | 1.2 |
| B2 | B1 | 1 | 2 | 1.2 | 0.2 | 1.2 |
| C2 | C1 | 1 | 2 | 1.2 | 0.2 | 1.2 |
| A3 | 3 | 1 | A2 | 1.2 | 0.2 | 1.2 |
| B3 | 3 | 1 | B2 | 1.2 | 0.2 | 1.2 |
| C3 | 3 | 1 | C2 | 1.2 | 0.2 | 1.2 |
| A4 | A3 | 1 | 2 | 1.2 | 0.2 | 1.2 |
| B4 | B3 | 1 | 2 | 1.2 | 0.2 | 1.2 |
| C4 | C3 | 1 | 2 | 1.2 | 0.2 | 1.2 |
| D1 | A1 | 1 | 2 | 0.5 | 0.2 | 1.2 |
| D2 | A1 | 1 | B2 | 1 | 0.2 | 1.2 |
| D3 | A1 | 1 | C2 | 1 | 0.2 | 1.2 |
| D4 | B1 | 1 | 2 | 0.5 | 0.2 | 1.2 |
| D5 | C1 | 1 | B2 | 1 | 0.2 | 1.2 |
| D6 | C1 | 1 | 2 | 0.5 | 0.2 | 1.2 |
| Q1 | A3 | 1 | 2 | 0.5 | 0.2 | 1.2 |
| Q2 | B3 | 1 | A4 | 1 | 0.2 | 1.2 |
| Q3 | C3 | 1 | A4 | 1 | 0.2 | 1.2 |
| Q4 | B3 | 1 | 2 | 0.5 | 0.2 | 1.2 |

SM: Starting material;
Acid 2: 1,18-Octadec-9-enedioic acid;
Alcohol 3: 2,3-dihydroxypropyl-3-oleyl glycerol;
DMAP: dimethylaminopyridine;
DCC: N,N′-dicyclohexylcarbodiimide

### Analytical Characterization of the Dimers and Quatrimers

All the synthesized compounds including the intermediates were characterized by ¹H-NMR. The oligomers were also additionally characterized by ¹³C-NMR. To further confirm the structures, D1 and Q1, as representatives of the dimers and quatrimers respectively, were characterized by MS. The corresponding NMR data is provided in Table 5 below.

### Nuclear Magnetic Resonance (NMR)

¹H and ¹³C-NMR spectra were recorded on a Bruker Avance III 400 spectrometer (Bruker BioSpin MRI GmbH, Karlsruhe, Germany) at a frequency of 400 MHz and 100 MHz respectively, using a 5-mm BBO probe. The 1D ¹H-NMR Spectra were acquired at 25° C. over a 16-ppm spectral window with a 1 second recycle delay, and 32 transients. The 1D ¹³C-NMR spectra were acquired at 25° C. over a 240-ppm spectral window with a 0.2 s recycle delay, and 2048 transients. The spectra were Fourier transformed, phase corrected, and baseline corrected. Window functions were not applied prior to Fourier transformation. Chemical shifts were referenced relative to residual solvent peaks.

### Mass Spectrometry (MS)

Electrospray ionization mass spectrometry (ESI-MS) analysis was performed with a QStar XL quadrupole time-of-flight mass spectrometer (AB Sciex, Concord, ON) equipped with an ionspray source and a modified hot source-induced desolvation (HSID) interfaces (Ionics, Bolton, ON). The ion source and interface conditions were adjusted as follows: ionspray voltage (IS)=4500 V, nebulizing gas (GS1)=45, curtain gas (G52)=45, declustering potential (DP)=60 V and HSID temperature (T)=200° C. Multiple-charged ion signals were reconstructed using the BioTools 1.1.5 software package (AB Sciex, Concord, ON).

### Gel Permeation Chromatography (GPC)

Gel permeation chromatography (GPC) was carried out on a Waters e2695 HPLC (Waters Limited, Mississauga, Ontario) fitted with a Waters e2695 pump, Waters 2414 refractive index detector and a Styragel HR5E column (5 μm). Chloroform was used as eluent with a flow rate of 1 mL/min. The sample was made with a concentration of 4 mg/mL, and the injection volume was 30 μL. Polystyrene (PS) standards were used to calibrate the curve.

### Thermogravimetric Analysis (TGA)

The measurements were carried out in triplicate on a Q500 TGA model (TA Instruments, DE, USA). Approximately 8.0-15.0 mg of fully melted and homogenously mixed sample was loaded in the open TGA platinum pan. The sample was equilibrated at 25° C. and heated to 600° C. at a constant rate of 3° C./min. The TGA measurements were performed under dry nitrogen of 40 mL/min for balance purge flow and 60 mL/min for sample purge flow. The onset temperature of degradation (T_{On}^{d}) was determined at the intersection of the baseline (0% weight loss line) and the tangent at the first inflexion point. The temperatures at 5% and 10% weight loss (T_{5%} and T_{10%}, respectively) were also used to assess the thermal stability of the samples. The derivative of the TGA (DTG) was used to determine the rate of degradation and the degradation steps, with the peak temperatures (T_{DTG}) signaling the maximum rate of degradation of each step.

### Differential Scanning Calorimetry (DSC)

The thermal measurements were carried out on a Q200 model DSC (TA Instruments, New Castle, Del., USA) equipped with a refrigerated cooling system (RCS 90, TA Instruments) under a nitrogen flow of 50 mL/min. The sample (5.0-6.0 (±0.1) mg), contained in a hermetically sealed aluminum pan was cooled from the melt (50° C.) to −90° C. and subsequently reheated to 70° C. at the same constant rate of 3.0° C./min to obtain the crystallization and melting profiles, respectively. TA Universal Analysis software was used to analyze the data and extract the main characteristics of the peaks. The measurement temperatures are reported to ±0.5° C.

### ¹H-NMR Results

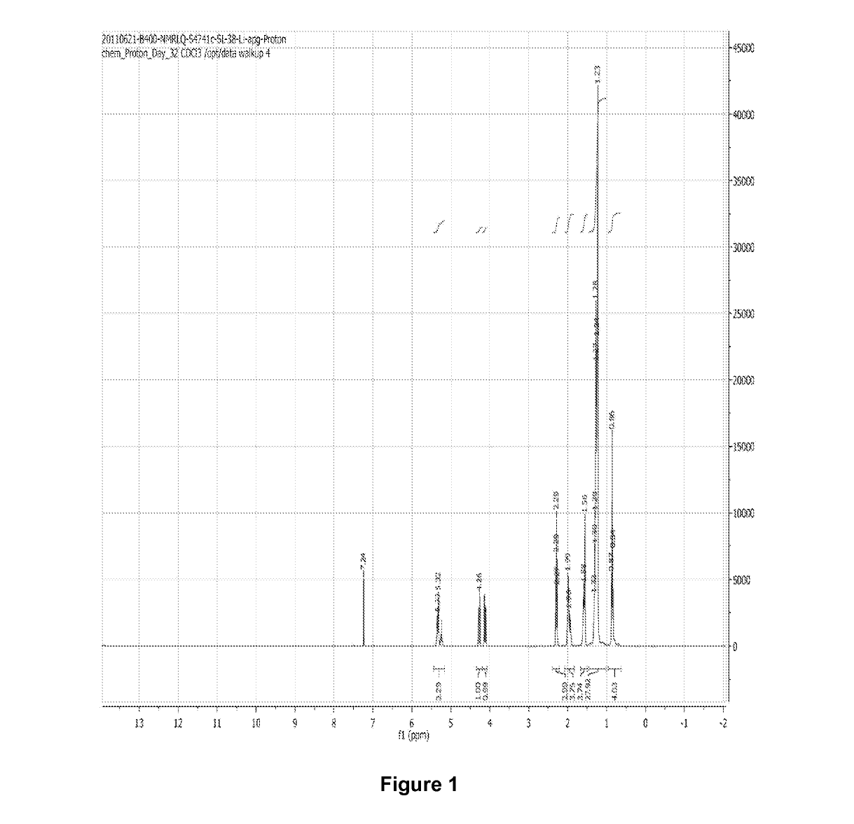

The ¹H-NMR spectrum of a representative dimer, is shown in FIG. 1. The —CH═CH— is presented at 5.38-5.32 ppm; —CH₂CH(O)CH₂— and —OCH₂CHCH₂O— on the glycerol skeleton at 5.27-5.25 ppm and 4.32-4.12 ppm, respectively; C(═O)CH₂— at 2.33-2.28 ppm, —CH₂CH═CH at 2.03-1.98 ppm, C(═O)CH₂CH₂— at 1.60 ppm, and —CH₃ at 0.88 ppm. The ratios of protons corresponding to —CH═CH—, O═CCH₂— and —CH₃ (Table 6) were used to identify the structure of the oligomers. As may be seen, the values obtained from the experimental data (ExD in Table 4) matched the values calculated from the chemical formulas (ThD in Table 4) of the oligomers.

As illustrated with the ¹H-NMR spectrum in FIG. 1, the ¹H-NMR data indicates that the oligomers contained double bonds in both the trans- and cis-configurations. Based on ¹H-NMR of reference materials triolein (100% cis) and trielaidin (100% trans), the chemical shifts 6 at 5.38-5.36 ppm, and at 5.36-5.32 ppm were assigned to the trans- and cis-geometries, respectively. The trans-configuration was attributed to 1,18-octadec-9-enedioic acid between the glycerol molecules, and the cis-configuration to the oleic acid. Only trans-geometry was found in D6, due the trans-configuration of 1,18-octadec-9-enedioic acid and absence of oleic acid during the preparation of this compound.

TABLE 4
Proton's ratio of the oligomers. The ratio is calculated based on the
proton amount of —CH═CH— estimated by its relative integrated ¹H-NMR shift.

| | CH═CH | | —CH₂C(═O)— | | —CH₂CH═ | | —CH₃ | |
|---|---|---|---|---|---|---|---|---|
| Compound | ExD | ThD | ExD | ThD | ExD | ThD | ExD | ThD |
| D1 | 1 | 1 | 1.3 | 1.2 | 2.1 | 2.0 | 1.2 | 1.2 |
| D2 | 1 | 1 | 1.6 | 1.5 | 2.0 | 2.0 | 1.57 | 1.5 |
| D3 | 1 | 1 | 2.1 | 2.0 | 2.0 | 2.0 | 2.6 | 2.0 |
| D4 | 1 | 1 | 2.1 | 2.0 | 2.1 | 2.0 | 2.3 | 2.0 |
| D5 | 1 | 1 | 3.1 | 3.0 | 2.0 | 2.0 | 3.0 | 3.0 |
| D6 | 1 | 1 | 6.2 | 6.0 | 2.1 | 2.0 | 5.7 | 6.0 |
| Q1 | 1 | 1 | 1.3 | | 2.0 | | 1.1 | |
| Q2 | 1 | 1 | 1.6 | 1.5 | 2.1 | 2.0 | 1.2 | 1.1 |
| Q3 | 1 | 1 | 1.8 | 1.7 | 2.1 | 2.0 | 1.4 | 1.3 |
| Q4 | 1 | 1 | 1.8 | 1.7 | 2.0 | 2.0 | 1.4 | 1.3 |

ThD: Theoretical value based on the molecular formula; ExD experimental value obtained from ¹H-NMR data.

### ¹³C-NMR Results

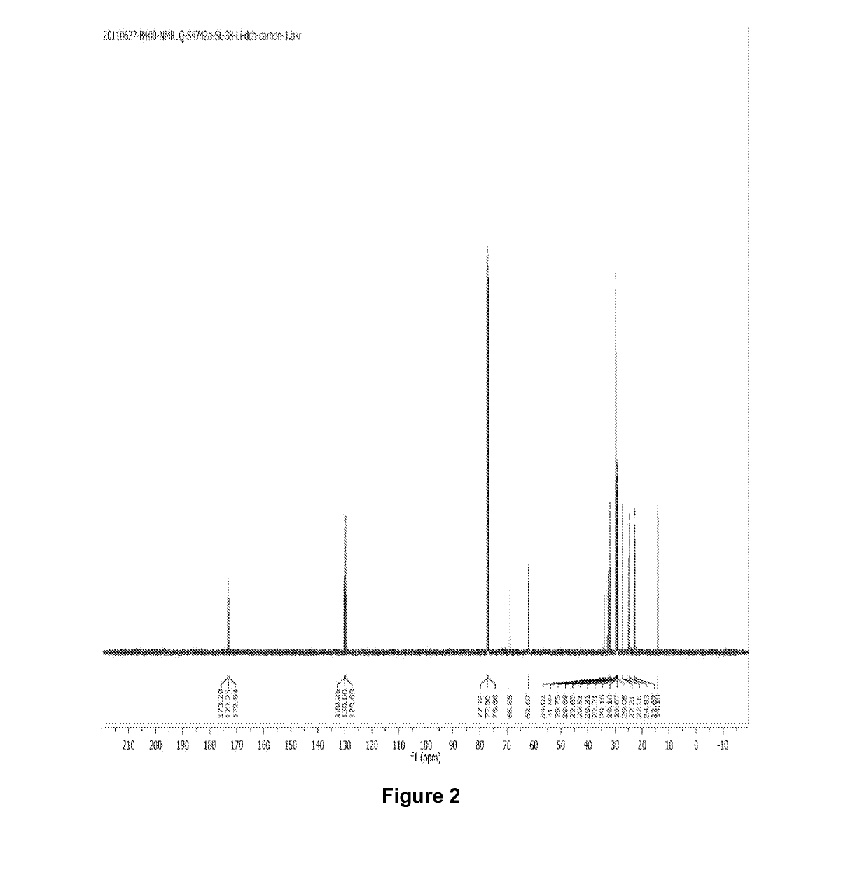

The ¹³C-NMR spectrum of D2 (refer to Table 6 for nomenclature) as an example of a NMR spectrum of the synthesized oligomers is shown in FIG. 2. The corresponding chemical shifts are provided in Table 5 below. TAG carbonyl carbons, alkenyl carbons, glyceryl carbons, and alkyl carbons were clearly identified in the ¹³C-NMR of the oligomers. Chemical shift due to α- and β-TAG carbonyl carbons showed at δ 173.45 ppm and 173.04 ppm, respectively, in the ¹³C-NMR spectra of the oligomers (FIG. 2). The glyceryl carbon at position sn-2 on the glycerol skeleton and at position sn-1(3) presented chemical shifts at δ 69.09 ppm and δ 62.31 ppm, respectively, in good agreement with the literature. Except D6, where the cis-configuration was absent, the oligomers presented both cis- (between 6=129 and 130.0 ppm) and trans-olefinic carbons (between δ 130 and 131 ppm), in agreement with previously reported data for the trans- and cis-configurations.

TABLE 5
NMR Data (start)

Synthesis of 2,3-dihydroxypropyl compounds
(2,2-dimethyl-1,3-dioxolan-4-yl)methyl oleate
¹H-NMR (in CDCl₃, ppm): δ = 5.38-5.30 (2H, m, —CH═CH—); 4.34-4.29 (1H, q,
—OCH₂CH(OH)—), 4.18 (1H, dd, —OCH₂CHCH₂O—), 4.16-4.08 (2H, m, —OCH₂CHCH₂O—), 3.73
(1H, dd, —OCH₂CHCH₂O—); 2.36-2.32 (2H, t, —OC(═O)CH₂CH₂—), 2.03-1.99 (4H, m,
—CH₂CH═CHCH₂—); 1.64-1.60 (2H, m, —OC(═O)CH₂CH₂—); 1.43 (3H, s, —CH₃); 1.37 (3H, s,
—CH₃); 1.34-1.27 (20H, m, —CH₂—), 0.90-0.86 (3H, t, —CH₃).
2,3-dihydroxypropyl oleate
¹H-NMR (in CDCl₃, ppm) δ = 5.4 (2H, m, —CH═CH—), 4.2-4.0 (5H, m, —OCH₂CH(OH)— +
—OCH₂CHCH₂O—), 2.4 (4H, t, —OC(═O)CH₂CH₂—), 2.0 (8H, m, —CH₂CH═CHCH₂—), 1.6 (4H, m,
—OC(═O)CH₂CH₂—), 1.4-1.2 (40H, m, —(CH₂)—), 0.9 (6H, t, —CH₃).
Synthesis of 1,3-disubstituted glycerol (A1, B1 or C1)
1,3-Dioleyloxypropan-2-one
¹H-NMR (in CDCl₃, ppm): δ = 5.39-5.30 (4H, m, —CH═CH—), 4.72 (4H, s, —OCH₂CH(OH)—),
2.44-2.40 (4H, t, —OC(═O)CH₂CH₂—), 2.03-1.98 (8H, m, —CH₂CH═CHCH₂—), 1.68-1.62
(4H, m, —OC(═O)CH2CH2—), 1.31-1.25 (40H, m, —CH₂—), 0.89-0.86 (6H, t, —CH₃)
1,3-Distearoyloxypropan-2-one
¹H-NMR (in CDCl₃, ppm): 4.75 (4H, S, —OCH₂CH(OH)—); 2.44-2.40 (4H, t, —OC(═O)CH₂CH₂—);
1.69-1.65(4H, m, —OC(═O)CH₂CH₂—); 1.40-1.20 (56H, m, —CH₂—), 0.89-0.87 (6H, t, —CH₃).
A1
1H-NMR (in CDCl3, ppm): 5.34 (4H, m, —CH═CH—); 4.18-4.14 (5H, m,
—OCH2CH(OH)— + —OCH2CHCH2O—); 2.36-2.33 (4H, t, —OC(═O)CH2CH2—); 2.02-2.00
(8H, t, —CH2CH═CHCH2—); 1.62 (4H, m, —OC(═O)CH2CH2—); 1.34-1.26 (40H, m,
—CH2—); 0.88 (6H, t, —CH3)
B1
1H-NMR (in CDCl3, ppm): 4.17-4.14 (4H, m, —OCH2CH(OH)—); 4.12-4.05 (1H, m,
—OCH2CHCH2O—); 2.42 (1H, t, —OH); 2.35-2.32 (4H, t , —OC(═O)CH2CH2—); 1.64-1.61
(4H, m, —OC(═O)CH2CH2—); 1.29-1.25 (56H, m, —CH2—), 0.89 (6H, t, —CH3)
C1
1H-NMR (in CDCl3, ppm): 5.34-5.31 (2H, m, —CH═CH—), 4.19-4.12 (4H, m,
—OCH2CH(OH)—), 4.09-4.05 (1H, m, —OCH2CHCH2O—), 2.44 (1H, br, —OH), 2.34-
2.31 (4H, t, —OC(═O)CH2CH2—), 2.01-1.96 (4H, m,—CH2CH═CHCH2—), 1.16-1.59
(4H, m, —OC(═O)CH2CH2—), 1.32-1.23 (54H, m, —CH2—), 0.87-0.84 (6H, t, —CH3).
Synthesis of 1,18-Octadec-9-enedioic acid (2)
¹H-NMR (in DMSO-d6, ppm): 11.94 (2H, s, —COOH), 5.36 (2H, t, —CH═CH—), 2.19-2.16 (4H,
m, —OC(═O)CH₂CH₂—), 1.94 (4H, m, —CH₂CH═CHCH₂—), 1.49-1.45 (4H, m, —OC(═O)CH₂CH₂—),
1.31-1.26 (18H, m, —CH₂—)
Route for synthesis of mono-acids (A2, B2 or C2)
A2
¹H-NMR (in CDCl₃, ppm): 5.38-5.32 (6H, m, —CH═CH—), 5.31-5.25 (1H, m, OCH₂CHCH₂O—),
4.31-4.27 (2H, dd, —OCH₂CH(OH)—), 4.16-4.11 (2H, dd, —OCH₂CH(OH)—), 2.36-2.28 (8H, t,
—OC(═O)CH₂CH₂—), 2.04-1.96 (12H, m, —CH₂CH═CHCH₂—), 1.63-1.59 (8H, t,
—OC(═O)CH₂CH₂—), 1.30-1.24 (56H, m, —CH₂—), 0.89-0.86 (6H, t, —CH₃)
B2
¹H-NMR (in CDCl₃, ppm): 5.36-5.34 (2H, t, —CH═CH—), 5.26-5.22 (1H, m,
—OCH₂CH(O)CH₂O—), 4.29-4.25 (2H, dd, —OCH₂CH(O)CH₂O—), 4.15-4.10 (2H, dd,
—OCH₂CH(O)CH₂O—), 2.31-2.27 (8H, m, —CH₂COO—), 2.03-1.92 (4H, m, —CH₂CH═), 1.61-1.56
(8H, m, —CH₂CH₂COO—), 1.32-1.23 (72, m, —CH₂—), 0.88-0.83 (6H, t, —CH₃).
C2
¹H-NMR (in CDCl₃, ppm): 5.40-5.36 (4H, m, —CH═CH—), 5.30-5.28 (1H, m,
—OCH₂CH(O)CH₂O—), 4.34-4.30 (2H, dd, —OCH₂CH(O)CH₂O—), 4.19-4.15 (2H, dd,
—OCH₂CH(O)CH₂O—), 2.39-2.32 (8H, t, —CH₂COO—), 2.04-1.98 (8H, m, —CH₂C═CH—), 1.67-
1.62 (8H, m, —CH₂CH₂COO—), 1.32-1.28 (64, m, —CH₂—), 0.92-0.89 (6H, t, —CH₃)
Synthesis of Dimers
D1
¹H-NMR (in CDCl₃, ppm): 5.35-5.31 (10H, m, —CH═CH—), 5.26-5.24 (2H, m,
—OCH₂CH(O)CH₂O—), 4.29-4.25(4H, dd, —CH(O)CH₂O—), 4.14-4.10 (4H, dd, —CH(O)CH₂O—),
2.31-2.27 (12H, t, —OOCCH₂—), 1.99-1.94 (20H, m, —CH═CHCH₂—), 1.61-1.57 (12H, m,
—OOCCH₂CH₂—), 1.28-1.25(102H, m, —CH2—), 0.88-0.86 (12H, t, —CH₃).
¹³C-NMR (in CDCl₃, ppm): 173.47, 173.06, 130.52, 130.23, 129.93, 69.10, 62.31, 34.42,
34.26, 32.82, 32.13, 29.99-29.29, 27.45, 27.40, 25.12, 25.07, 22.91, 14.34;
MS, C₉₆H₁₇₂O₁₂, Cal. 1518.39, found 1541[M + Na]+
D2
¹H-NMR(in CDCl₃, ppm): 5.36-5.30 (8H, m, —CH═CH—), 5.26 (2H, m, —OCH₂CH(O)CH₂O—),
4.31-4.27 (4H, dd, —CH(O)CH₂O—), 4.16-4.12 (4H, dd, —CH(O)CH₂O—), 2.31-2.27 (12H, t,
—OOCCH₂—), 2.01-1.92 (16H, m, —CH═CHCH₂—), 1.63-1.60 (12H, m, —OOCCH₂CH₂—), 1.30-
1.27 (108H, m, —CH₂—), 0.90-0.86 (12H, t, —CH₃);
¹³C-NMR(in CDCl₃, ppm): 173.29, 172.84, 130.26, 130.00, 129.69, 68.85, 62.07, 34.01,
31.89, 29.75, 29.69, 29.65-29.05, 27.21, 27.16, 24.83, 22.67, 14.10
D3
¹H-NMR(in CDCl₃, ppm): 5.36-5.30 (6H, m, —CH═CH—), 5.24 (2H, m, —OCH₂CH(O)CH₂O—),
4.31-4.27 (4H, dd, —CH(O)CH₂O—), 4.16-4.12 (4H, dd, —CH(O)CH₂O—), 2.31-2.27 (12H, t,
—OOCCH₂—), 2.01-1.92 (12H, m, —CH═CHCH₂—), 1.63-1.60 (12H, m, —OOCCH₂CH₂—), 1.30-
1.27 (114H, m, —CH₂—), 0.90-0.86 (12H, t, —CH₃);
¹³C-NMR(in CDCl₃, ppm): 173.28, 172.84, 130.26, 129.99 129.69, 68.85, 62.06, 34.01,
31.91, 29.75, 29.69, 29.65-29.04, 27.20, 27.15, 24.85, 22.67, 14.10
¹³C-NMR (in CDCl₃ ppm): 173.40, 172.99, 130.63, 130.30, 69.11, 26.33, 34.43, 34.26,
32.82, 32.13, 31.15, 29.89-29.20, 25.10, 22.91, 14.34
D4
¹H-NMR (in CDCl₃, ppm): 5.36-5.30 (6H, m, —CH═CH—), 5.24 (2H, m, —OCH₂CH(O)CH₂O—),
4.28-4.25 (4H, dd, —CH(O)CH₂O—), 4.14-4.10 (4H, dd, —CH(O)CH₂O—), 2.31-2.27 (12H, t,
—OOCCH₂—), 2.01-1.92 (12H, m, —CH═CHCH₂—), 1.63-1.60 (12H, m, —OOCCH₂CH₂—), 1.30-
1.27 (114H, m, —CH₂—), 0.90-0.86 (12H, t, —CH₃);
¹³C-NMR (in CDCl₃, ppm): 173.28, 172.84, 130.26, 129.99, 129.69, 68.85, 62.07, 34.18,
34.03, 34.03, 32.58, 31.91, 29.75, 29.69, 29.65-29.05, 27.20, 27.16, 24.85, 22.67, 14.10
D5
¹H-NMR (in CDCl₃, ppm): 5.38-5.31(4H, m, —CH═CH—), 5.26-5.21 (2H, m,
—OCH₂CH(O)CH₂O—), 4.29-4.25 (4H, dd, —CH(O)CH₂O—), 4.14-4.10 (4H, dd, —CH(O)CH₂O—),
2.31-2.27 (12, t, —OOCCH₂—), 2.00-1.93 (8H, m, —CH═CHCH₂—), 1.59 (12H, m,
—OOCCH₂CH₂—), 1.27-1.23 (120H, m, —CH₂—), 0.88-0.84 (12H, t, —CH₃).
¹³C-NMR (in CDCl₃, ppm): 173.40, 172.99, 130.63, 130.30, 69.11, 26.33, 34.43, 34.26,
32.82, 32.13, 31.15, 29.89-29.20, 25.10, 22.91, 14.34
D6
¹H-NMR (in CDCl₃, ppm): 5.36-5.35 (2H, m, —CH═CH—), 5.25-5.23 (2H, m,
—OCH₂CH(O)CH₂O—), 4.29-4.25 (4H, dd, —CH(O)CH₂O—), 4.14-4.11 (4H, dd, —CH(O)CH₂O—),
2.30-2.27 (12H, t, —OOCCH₂—), 1.94-1.93 (4H, br, —CH═CHCH₂—), 1.60-1.58 (16H, m,
—OOCCH₂CH₂—), 1.27-1.23 (126, m, —CH₂—), 0.87-0.84 (6H, m, —CH₃)
¹³C-NMR (in CDCl₃, ppm): 173.50, 173.06, 130.49, 69.09, 62-30, 34.28, 32.83, 32.16,
29.93-29.29, 25.13, 25.10, 22.92, 14.35
Synthesis of A3, B3 or C3
A3
¹H-NMR (in CDCl₃, ppm): 5.36-5.34 (8H, m, —CH═CH—), 5.33-5.31 (1H, m,
—OCH₂CH(O)CH₂O—), 4.29-4.22 (4H, dd, —CH(O)CH₂O—), 4.18-4.05 (5H, m, —CH(O)CH₂O— +
—OCH₂CH(O)CH₂O—), 2.45-2.27 (10H, m, —CH₂COO—), 2.01-1.91 (16H, m, —CH₂CH═), 1.63-
1.56 (10H, m, —CH₂CH₂COO—), 1.32-1.23 (76H, m, —CH₂—), 0.88-0.81 (9H, m, —CH₃)
B3
¹H-NMR (in CDCl₃, ppm): 5.36-5.32 (6H, m, —CH═CH— + —OCH₂CH(O)CH₂O—), 4.29-4.25
(4H, dd, —CH(O)CH₂O—), 4.17-4.09 (5H, m, —CH(O)CH₂O— + —OCH₂CH(O)CH₂O—), 2.34-2.27
(10H, m, —CH₂COO—), 2.15 (1H, s, —OH), 2.02-1.93 (8H, m, —CH₂CH═), 1.61-1.56 (10H, m,
—CH₂CH₂COO—), 1.30-1.23(91H, m, —CH₂—), 0.87-0.84 (9H, t, —CH₃)
C3
¹H-NMR (in CDCl₃, ppm): 5.40-5.36 (6H, m, —CH═CH—), 5.30-5.28 (1H, m,
—OCH₂CH(O)CH₂O—), 4.34-4.30 (4H, m, —CH(O)CH₂O—), 4.20-4.12 (5H, m, —CH(O)CH₂O— +
—OCH₂CH(O)CH₂O—), 2.39-2.32 (10H, m, —CH₂COO—), 2.08-1.98 (12H, m, —CH₂CH═), 1.65-
1.62 (10H, m, —CH₂CH₂COO—), 1.33-1.28 (85H, m, —CH₂—), 0.93-0.89 (9H, t, —CH₃).
Synthesis of A4, B4 or C4
A4
¹H-NMR (in CDCl₃, ppm): 5.38-5.30 (10H, m, —CH═CH—), 5.25-5.23 (2H, m,
—OCH₂CH(O)CH₂O—), 4.29-4.25 (4H, dd, —CH(O)CH₂O—), 4.14-4.10 (4H, dd, —CH(O)CH₂O—),
2.34-2.27 (14H, m, —OOCCH₂—), 2.02-1.94 (20H, m, —CH═CHCH₂—), 1.60-1.57 (14H, m,
—OOCCH2CH2—), 1.28-1.24 (92H, m, —CH₂—), 0.88-0.81 (9H, t, —CH₃)
B4
¹H-NMR (in CDCl₃, ppm): 5.36-5.32 (8H, m, —CH═CH— + —OCH₂CH(O)CH₂O—), 4.29-4.25 (4H,
dd, —CH(O)CH₂O—), 4.15-4.10 (4H, dd, —CH(O)CH₂O—), 2.32-2.27 (12H, m, —OOCCH₂—),
2.03-1.91 (12H, m, —CH═CHCH₂—), 1.61-1.56 (12H, m, —OOCCH₂CH₂—), 1.32-1.23 (112H,
m, —CH₂—), 0.87-0.84 (9H, t, —CH₃)
C4
¹H-NMR (in CDCl₃, ppm): 5.40-5.36 (8H, m, —CH═CH—), 5.30-5.28 (2H, m, —CH₂CH(O)CH₂—),
4.34-4.30 (4H, dd, —CH(O)CH₂O—), 4.19-4.15 (4H, dd, —CH(O)CH₂O—), 2.39-2.32 (12H, m,
—OOCCH₂—), 2.04-1.98 (16H, m, —CH═CHCH₂—), 1.67-1.62(12H, m, —OOCCH₂CH₂—), 1.32-
1.28 (104H, m, —CH₂—), 0.92-0.89 (9H, t, —CH₃)
Synthesis of Quatrimers
Q1
¹H-NMR (in CDCl₃, ppm): 5.38-5.33 (18H, m, —CH═CH—), 5.27-5.25 (4H, m,
—OCH₂CH(O)CH₂O—), 4.31-4.27 (8H, dd, —CH(O)CH₂O—), 4.16-4.12 (8H, dd, —CH(O)CH₂O—),
2.33-2.29 (24H, t, —OOCCH₂—), 2.03-1.93 (36H, m, —CH═CHCH₂—), 1.62-1.60 (24H, m,
—OOCCH₂CH₂—), 1.30-1.26 (168H, m, —CH₂—), 0.90-0.86 (18H, t, —CH₃)
¹³C-NMR (in CDCl₃, ppm): 173.47, 173.06, 130.52, 130.23, 129.93, 69.09, 62.31, 34.41,
34.25, 32.81, 32.13, 29.99-29.29, 27.44, 27.39, 25.11, 25.06, 22.90, 14.33
MS, C₁₇₄H₃₀8O₂₄, Cal. 2784.29, found 2783(M)^{⁺}, 2802.4 [M + NH4]^{⁺}
Q2
¹H-NMR(in CDCl₃, ppm): 5.36-5.30 (16H, m, —CH═CH—), 5.24 (4H, m, —OCH₂CH(O)CH₂O—),
4.29-4.24 (8H, dd, —CH(O)CH₂O—), 4.14-4.09 (8H, dd, —CH(O)CH₂O—), 2.31-2.27 (24H, t,
—OOCCH₂—), 2.02-1.91 (32H, m, —CH═CHCH₂—), 1.63-1.60 (24H, m, —OOCCH₂CH₂—), 1.32-
1.23 (178H, m, —CH₂—), 0.87-0.84 (18H, t, —CH₃);
¹³C-NMR (in CDCl₃, ppm): 173.48, 173.07, 130.52, 130.24, 129.93, 69.10, 62.32, 34.43,
34.26, 32.83, 32.16, 30.00-29.30, 27.46, 25.12, 22.92, 14.35
Q3
¹H-NMR (in CDCl₃, ppm): δ = 5.36-5.30 (14H, m, —CH═CH—), 5.24 (4H, m, —OCH₂CH(O)CH₂O—),
4.31-4.27 (8H, dd, —CH(O)CH₂O—), 4.16-4.12 (8H, dd, —CH(O)CH₂O—), 2.31-2.27 (24H, t,
—OOCCH₂—), 2.02-1.91 (28H, m, —CH═CHCH₂—), 1.63-1.60 (24H, m, —OOCCH₂CH₂—), 1.32-1.23
(186H, m, —CH₂—), 0.87-0.84 (18H, t, —CH₃);
¹³C-NMR (in CDCl₃, ppm): 173.48, 173.07, 130.50, 130.24, 129.94, 69.10, 62.31, 34.43,
32.83, 32.17, 30.01-29.30, 27.46, 25.13 22.92, 14.36
Q4
¹H-NMR(in CDCl₃, ppm): 5.37-5.30 (14H, m, —CH═CH—), 5.24 (4H, m, —OCH₂CH(O)CH₂O—),
4.29-4.25 (8H, dd, —CH(O)CH₂O—), 4.14-4.10 (8H, dd, —CH(O)CH₂O—), 2.30-2.27 (24H, t,
—OOCCH₂—), 1.99-1.94 (28H, m, —CH═CHCH₂—), 1.60-1.57 (24H, m, —OOCCH₂CH₂—), 1.32-1.23
(186H, m, —CH₂—), 0.87-0.84 (18H, t, —CH₃);
¹³C-NMR (in CDCl₃, ppm): 173.47, 173.06, 130.49, 130.23, 129.93, 69.10, 62.31, 34.42,
34.26, 32.82, 32.16, 30.00-29.29, 27.45, 25.12 22.91, 14.34

NMR Data (end)

### Physical Properties of the Dimers and Quatrimers

### Saturation

The relative number of “straight” fatty chains was found to be the optimal structural indicator of the variation of the thermal properties of the dimers and quatrimers. This variable was calculated as the ratio between the number of “straight” fatty chains, i.e., the saturated and the trans-fatty chains and the total number of fatty acid chains in the oligomer. The trans-fatty chains are found only in the bridge between the terminal glycerols of the oligomers (one trans-fatty chain in the dimers and three trans-fatty chain in the quatrimers, see Scheme 1). This variable is referred to as the level of saturation, or simply saturation, and is calculated in percent. The saturation values obtained for the oligomers are listed in Table 6 below. The position of the fatty acid chains in the molecule does not factor into the measurement of saturation.

TABLE 6
Structural data of the dinners (D) and quatrimers (Q).

| | | | | | | | | | | About | About |
|---|---|---|---|---|---|---|---|---|---|---|---|
| Compound | | #Carbons | Mw | R₁—COO | R₂—COO | R₃—COO | R₄—COO | R₅—COO | % Sat | Bridge | Center |
| Dimer 1 | D1 | 96 | 1518 | OA | OA | OA | OA | — | 20 | S | S |
| Dimer 2 | D2 | 96 | 1520 | OA | OA | OA | SA | — | 40 | A | A |
| Dimer 3 | D3 | 96 | 1522 | OA | OA | SA | SA | — | 60 | A | S |
| Dimer 4 | D4 | 96 | 1522 | OA | SA | OA | SA | — | 60 | S | A |
| Dimer 5 | D5 | 96 | 1524 | OA | SA | SA | SA | — | 80 | A | A |
| Dimer 6 | D6 | 96 | 1528 | SA | SA | SA | SA | — | 100 | S | S |
| Quatrimer 1 | Q1 | 174 | 2784 | OA | OA | OA | OA | OA | 33 | S | S |
| Quatrimer 2 | Q2 | 174 | 2786 | OA | SA | OA | OA | OA | 44 | A | A |
| Quatrimer 3 | Q3 | 174 | 2788 | OA | OA | SA | SA | OA | 56 | A | S |
| Quatrimer 4 | Q4 | 174 | 2788 | OA | SA | OA | SA | OA | 56 | S | A |

OA: oleic acid anion;
SA: stearic acid anion;
Mw: Molecular weight;
% Sat: ratio of trans- plus saturated fatty acid chain number to the total number of fatty acid chains, referred to as saturation in the text.
Symmetry about the bridge and center: Symmetrical (S) and Asymmetrical (A).

### Thermal Stability

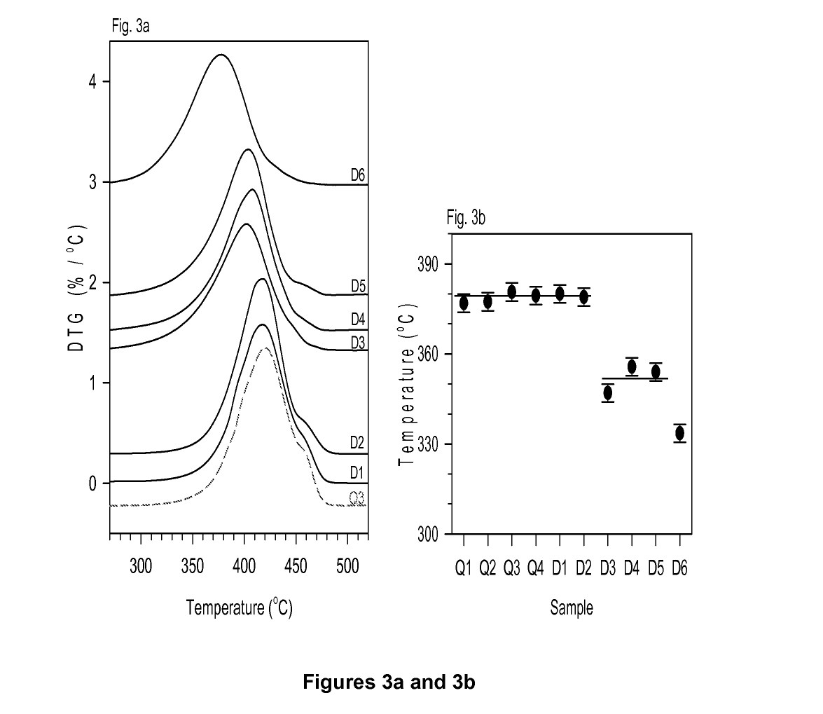

The derivatives of the TGA curves of the dimers and quatrimers are shown in FIG. 3a. The corresponding TGA data is listed in Table 7. The overall thermal degradation temperatures are relatively high (see Table 7) indicating that these classes of compounds demonstrate excellent thermal stability, better than common commercial vegetable oils, such as olive, canola, sunflower and soybean oils, for which first DTG peaks are presented at 325° C. For all the oligomers, eighty percent (80%) of sample mass was lost between T_{On}^{d} and ˜440° C., due to scissions at the ester groups level, and the remaining 20% was lost from ˜440 to 480° C., due to decomposition of the carbon chains and other fragments that may have been produced at high temperatures under the N₂ atmosphere.

The quatrimers all presented similar TGA/DTG profiles, indicating that their decomposition mechanism did not change with molecular variation. All quatrimers presented a T_{On}^{d}, at 379±2° C. The main DTG peak of the quatrimers (T_{DM} at 420±2° C.) is preceded by two shouldering peaks, revealing that the degradation of these compounds between T_{On}^{d}, and 440° C. involved three overlapping steps; the position of the ester group in quatrimers, such as at sn-1, sn-2, internal or outer of structure affected their thermal stability. The successive peaks observed in the DTG of the quatrimers indicates that decomposition initiated with a scission (T_{D1}) at the weakest position (the β-hydrogen) of the internal ester groups (R5 in Scheme 1), followed by scission (T_{D2}) at the β-hydrogen located at the sn-2 positions and then decomposition (T_{DM}) of the outer sn-1(3) ester groups.

The decomposition profiles of the dimers may be categorized into three groups. The first group is formed by D1 and D2. Their TGA and DTG profiles were almost similar to those of the quatrimers with exactly the same T_{On}^{d}, T_{10%} and T_{DM} (FIG. 3b) but with only one shoulder peak at 400±5° C. This peak corresponds to T_{D2} of the quatrimers. This is because the double bond content (less than 20%) in D1 and D2 is within the same range as in the quatrimers. The absence of T_{D1} in the DTG curves of the dimers suggests that T_{D1} is due to the decomposition of internal ester linkages, which supports the attribution above. When the content of the double bond decreases, the thermal stability also decreases, as seen in D3 to D6. The second group includes D3, D4 and D5 with all three dimers presenting effectively the same T_{On}^{d}, at 352±5° C. and T_{DM} at 405±3° C. D6 forms the third group with a decomposition profile that is different from all the others and presented one wide DTG peak (T_{DM} at 379±3° C.) and the lowest characteristic temperatures (T_{On}^{d}, and T_{10%}=330±3° C.).

The DTG peaks at T_{D2} and T_{DM} of the dimers are associated with the scission at the β-hydrogen located at the sn-2 and sn-1(3) of their ester groups, respectively. The weight loss at each step corresponded roughly to the mass ratios of the chains involved. The thermal stability of the dimers is mainly affected by the degree of unsaturation, and only slightly by the relative position of the unsaturation. The saturated dimer (D6) showed the lowest decomposition characteristics followed by the dimers of the second group that have one or two unsaturated fatty acid and the dimers of the first group that have three or four unsaturated fatty acids. The effect of a single unsaturated fatty acid cannot be determined in each of these groups, due to the insufficient deconvolution of the DTG curve. It is, however, clear that the neighboring unsaturated fatty acid chains enhance the thermal stability of the dimers in a stepwise manner as indicated by the discontinuous increase in the degradation temperatures (FIG. 3b). The data suggest that unsaturation imparts strength to its closest weakest link, i.e., the β-hydrogen at the sn-1(3) position.

TABLE 7
TGA data of the oligomer.

| | | Symmetry | | | | |
|---|---|---|---|---|---|---|
| Dinner/ | | About | About | | | |
| quatrimer | % saturation | Bridge | Center | T_{DM} | T_{10%} | Tₒₙ |
| Q1 | 33 | S | S | 420 | 371 | 377 |
| Q2 | 44 | A | A | 420 | 373 | 377 |
| Q3 | 56 | A | S | 421 | 380 | 381 |
| Q4 | 56 | S | A | 419 | 375 | 379 |
| D1 | 20 | S | S | 418 | 378 | 380 |
| D2 | 40 | A | A | 418 | 379 | 379 |
| D3 | 60 | A | S | 403 | 339 | 347 |
| D4 | 60 | S | A | 407 | 349 | 356 |
| D5 | 80 | A | A | 404 | 345 | 354 |
| D6 | 100 | S | S | 379 | 326 | 334 |

T_{DM}: Peak temperature of the main DTG peak; T_{On}^{d}: onset temperature of degradation determined at the intersection of the baseline (0% weight loss line) and the tangent at the first inflexion point.
T_{10%}: Temperatures at 10% weight loss.
% Sat: % Sat: ratio of trans- plus saturated fatty acid chain number to the total number of fatty acid chains, referred to as saturation in the text.
S: symmetrical and A: asymmetrical denote the symmetry about the bridge plane and about the center of the molecule.

### Crystallization and Melting Behaviors

### Crystallization Behavior

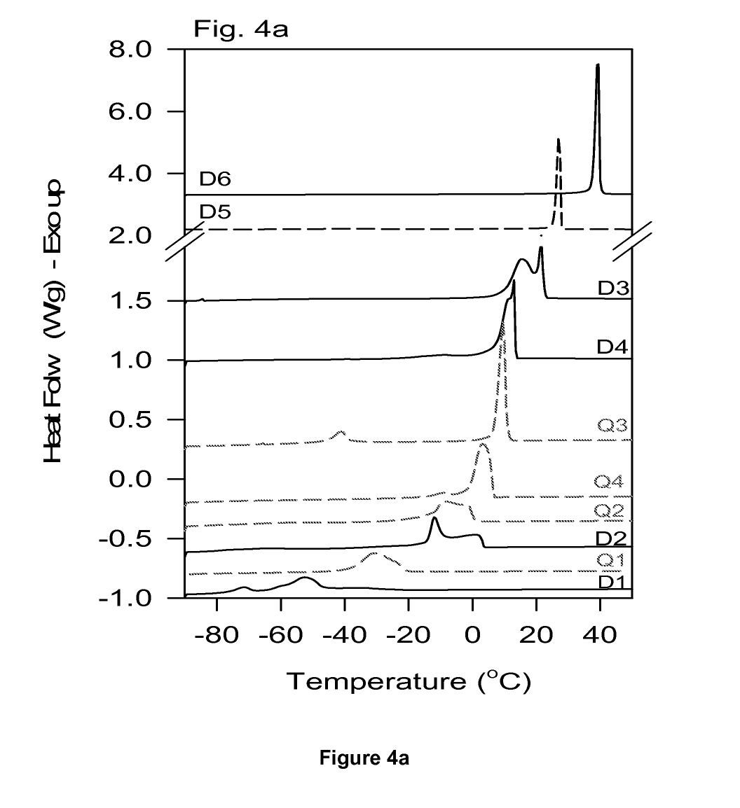

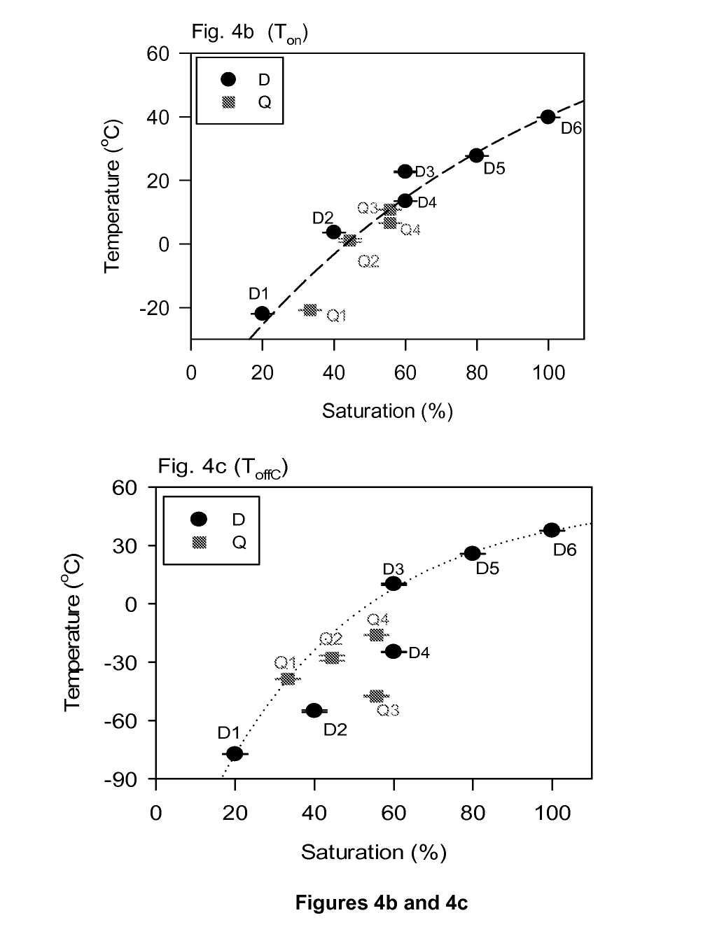

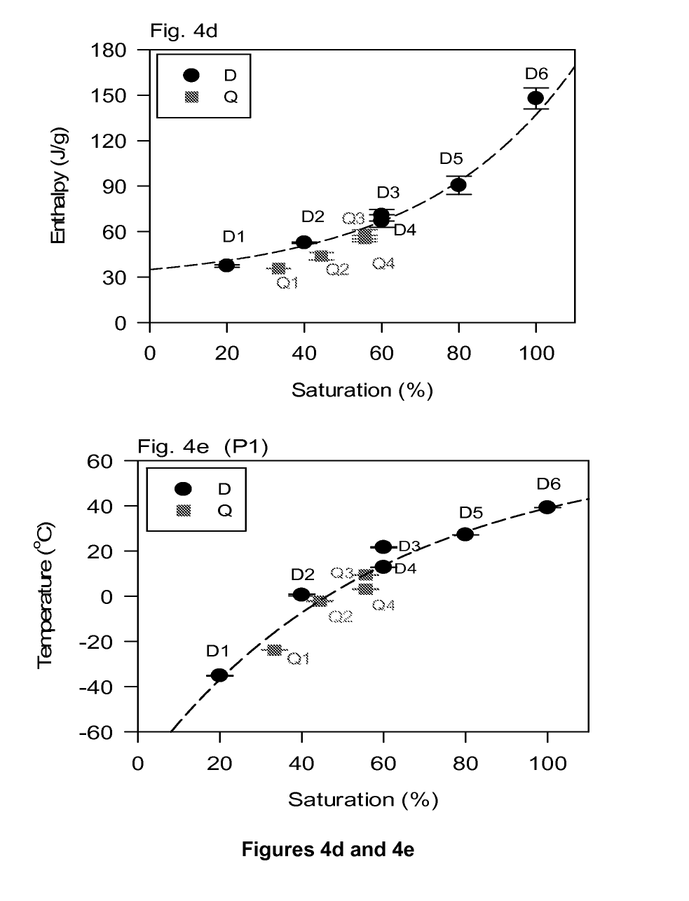

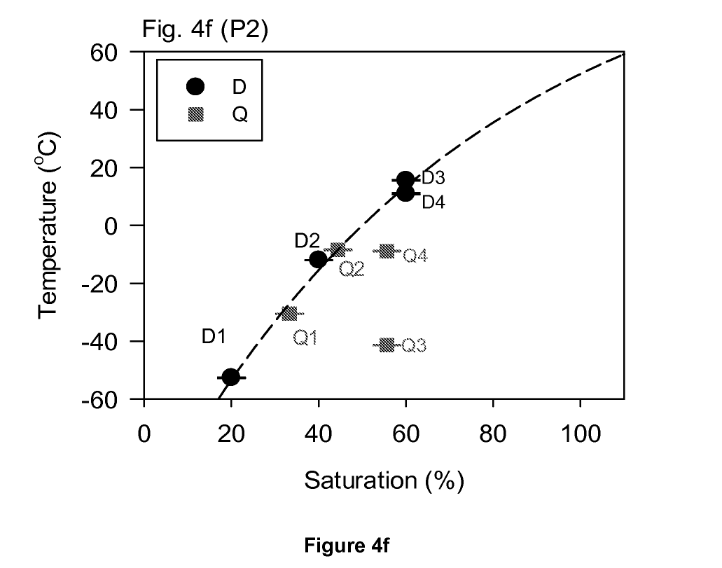

The cooling thermograms of the dimers and quatrimers are shown in FIG. 4a. The onset (Tₒₙ), offset temperature (T_{off}) and enthalpy of crystallization (ΔH_{C}) versus saturation curves are shown in FIGS. 4b, 4c and 4d, respectively. FIG. 4a highlights the noticeable shift of the DSC signal to higher temperatures as the level of saturation increases. The differences between the thermograms of oligomers with the same saturation but different distribution of the terminal fatty acids on the glycerols (D3 and D4, and Q3 and Q4) underscores the significant effect of symmetry.

The cooling thermograms of D1, D2 and D3 presented very low temperature transitions (VLTs, not shown) at −71, −64 and −46° C., respectively; whereas, D4, D5 and D6 did not. Q1, Q2, and Q3 also showed VLTs at −76, −54, and −41° C. and Q4 did not. The cooling thermograms of the oligomers with 80% saturation or more (D5 with one unsaturated fatty acid and the saturated D6) presented only one exotherm (P1 in FIG. 4a) and all the oligomers with lower saturation (D1, D2, D3 and D4 are the dimers with two or more unsaturated fatty acids and all the quatrimers) presented two well-defined exotherms (P1 and P2 in FIG. 4a) indicating either the formation of two different phases or a polymorphic transformation.

### Effect of Saturation Levels

The VLTs strongly depended on saturation. Their peaks shifted to higher temperatures, their widths increased, and associated enthalpies decreased noticeably as the number of unsaturated fatty acids decreased. This is understandable as these phases are affected by increased steric hindrances due to the presence of the kinked unsaturated fatty acids (four in D1, three in D2 and two in D3). The very low enthalpy measured for these phases (5.6 J/g for D1, 1.4 J/g for D2 and 0.3 J/g for D3) indicates that only very small portions of the material was involved in these transformations.

As may be seen in FIGS. 4a and 4b, the range of onset temperature of crystallization (Tₒₙ^{C}) available for the oligomers is very large. From −22° C. recorded for D1 (20% saturation), Tₒₙ^{C} increased almost linearly with increasing saturation to reach 40° C. for D6 (100% saturation). Barring the effect of symmetry and size, the offset temperature of crystallization (T_{off}^{C}) of the oligomers increased exponentially from −77° C. for the least saturated oligomers to 38° C. for the most saturated oligomer (dotted line in FIG. 4c), leading to increasingly shorter crystallization spans. The least saturated dimer (D1), for example, completed its crystallization over ˜55° C.; whereas, the most saturated dimer D6 and D5 crystallized in a temperature window of ˜2° C.

The total enthalpy of crystallization increased exponentially from a value as small as 37 J/g for D1 to 148 J/g for D6 (±up to 7.00 J/g), indicating very different phases and noticeably different propensity to form crystals. This is not surprising given that the symmetry of the molecule increases proportionally with saturation, leading to increased ease of packing.

The temperature at maximum height of P1 and P2 increased almost linearly with increasing saturation to reach a value of ˜60° C., indicating the increasing stability of the associated crystals. As the degree of saturation was increased, the height and enthalpy of the leading exotherm (P1) increased while those of P2 decreased, suggesting the competition of two different transformation processes. P1 is associated with the nucleation and growth of a phase that is established mainly by the trans- and saturated structural elements, and P2 is associated with either another phase or a polymorphic transformation that is driven by the unsaturated fatty acids of the oligomer. One may notice that for the dimers, (T_{P1}-T_{P2}) separation decreased noticeably from D1 to D4, after which only P1 was observed, outlining the competition between the saturated and unsaturated contributions to the overall molecular interactions. The substantial decrease of the full width at half maximum of P1 indicated that the disrupting effect of the unsaturated chains is minimized as saturation increases, leading to more homogeneous phases. This suggests that as saturation increases, polymorphic transformations are more likely than the nucleation of new phases.

It is worth noting that the characteristics parameters, such as Tₒₙ and T_{off}, etc., of the dimers and quatrimers (solid squares in FIG. 4b) adhere very well to predictive trends. The extent at which the crystallization path may be controlled is wide-ranging. The vast range of crystallization temperatures that one may reach by varying the degree of saturation of the oligomers is notable and provides prescriptive information for the custom engineering of a variety of usages.

The representative crystallization data of the dimers and quatrimers described herein shown in Table 8 below.

TABLE 8
Crystallization data of dimers and quatrimers obtained during the
cooling rate at 3° C./min. Tₒₙₛₑₜ, T_{offset}, Tₚ₁, and Tₚ₂ are
the temperature of onset, offset, Peak 1, and Peak 2, respectively.

| | Crystallization | | | | |
|---|---|---|---|---|---|
| Samples | Tₒₙₛₑₜ (° C.) | T_{offset} (° C.) | T_{P1} (° C.) | Tₚ₂ (° C.) | Enthalpy (J/g) |
| D1 | −22.00 ± 0.04 | −77.24 ± 0.08 | −35.11 ± 0.20 | −52.68 ± 0.19 | 37.42 ± 0.82 |
| D2 | 3.59 ± 0.04 | −55.10 ± 0.52 | 0.59 ± 0.43 | −11.94 ± 0.06 | 52.70 ± 0.55 |
| D3 | 22.65 ± 0.26 | 10.09 ± 0.44 | 21.66 ± 0.23 | 15.58 ± 0.22 | 70.80 ± 3.85 |
| D4 | 13.43 ± 0.05 | −24.71 ± 0.23 | 12.89 ± 0.09 | 11.01 ± 0.27 | 66.99 ± 4.07 |
| D5 | 27.68 ± 0.02 | 25.69 ± 0.13 | 27.23 ± 0.04 | — | 90.51 ± 6.00 |
| D6 | 39.84 ± 0.05 | 37.64 ± 0.08 | 39.26 ± 0.02 | — | 147.83 ± 7.00 |
| Q1 | −20.80 ± 0.14 | −38.59 ± 0.19 | −23.73 ± 0.13 | −30.53 ± 0.07 | 35.72 ± 0.18 |
| Q2 | 1.11 ± 0.59 | −27.86 ± 1.42 | −2.06 ± 0.17 | −8.47 ± 0.25 | 43.85 ± 2.44 |
| Q3 | 10.76 ± 0.12 | −47.51 ± 0.38 | 9.48 ± 0.06 | −41.38 ± 0.08 | 58.29 ± 3.05 |
| Q4 | 6.54 ± 0.02 | −16.02 ± 0.03 | 3.14 ± 0.13 | −8.86 ± 0.15 | 55.49 ± 2.05 |

### Effect of Symmetry

Although saturation was an important indicator, one may noticeably change the crystallization behavior simply by changing the symmetry of the molecule. Similar to TAGs, for which the effect of symmetry on physical properties is very well documented, geometrical configuration of the oligomers had a significant impact of the on their phase behavior. In fact, the steric hindrances increase with asymmetry about the sn-2 positions, and with asymmetry about the bridge plane and/or the center of the molecules.

For similar saturation levels, the crystallization parameters of the dimers and quatrimers of the present work depended significantly on the position of the fatty acids on the terminal glycerol molecules. For example, D4 did not show a VLT transition despite having the same number of unsaturated fatty acids as D3, due to the fact that D3 has its unsaturated fatty acids on one glycerol molecule and its saturated fatty acids on the other, introducing extra steric hindrances at one end compared to D4 that has its unsaturated and saturated fatty acids distributed on each of its glycerol molecules, preventing the formation of very low temperature phases.

The symmetry considerations about the center of the molecule and about the sn-2 positions of the glycerol backbones are at the source of the differences in thermal properties recorded for oligomers with the same saturation levels.

The strong effect of symmetry on the way these compounds organize may be appreciated in the large differences between the crystallization parameters of D3 and D4 as well as Q3 and Q4. D3 started crystallizing much earlier than D4 (22.7° C. compared to 13.4° C.). The main crystallization events completed at 10° C. in D3 and at −25° C. in D4. Although not very large, a difference in total enthalpy of crystallization was recorded between D3 and D4 revealing the effect of symmetry (FIG. 4d). The small difference in ΔH_{C} between D3 and D4 suggests that overall, the missing enthalpy in one of the two coexisting phases was counterbalanced by the extra enthalpy of the other. Similar differences in the location of the terminal fatty acids between Q3 and Q4 motivated similar differences in thermal properties, although with smaller magnitude due to the larger size of the quatrimers. For example, Q3 crystallized at a higher temperature than Q4 (Tₒₙ at 10.8 and 6.4° C., respectively). More importantly, Q4 displayed a fundamentally different crystallization path compared to Q3 (FIG. 4a). Q3 presented a strong narrow first exotherm (peak at 11° C. in FIG. 4a) followed by a well-defined transition at lower temperature (peak at −41° C. in FIG. 4a), indicating the nucleation and growth of two separate phase. Q4 presented a much wider first transition and completed its crystallization earlier than Q3, suggesting a polymorphic transformation and a further ordering of the crystals.

The symmetry about the center of the molecule determines the relative stability of the phases formed. For instance, the proximity of the bridge and stearic acids in the case of D3(Q3), provides prolonged saturated linear segments at one end of the molecule that may accommodate stronger contacts compared to the symmetrical D4(Q4) where the unsaturated and saturated fatty acids are distributed equally on the two glycerol molecules, preventing the formation of higher temperature phases. On the other hand, the symmetry about the bridge was the determining factor in driving the complexity of the transformations themselves. Although these symmetries are somewhat related, one may attribute the differences between the characteristic temperatures of crystallization of oligomers with similar saturation mainly to the symmetry about the center and the complexity of the transformation path mainly to the symmetry about the bridge.

Effect of Size

Although the terminal structures of D3 and D4 were similar those of Q3 and Q4, respectively (similar versus mixed fatty acids at the glycerol molecules, see Scheme 3), Tₒₙ^{C}, was affected much more strongly in the dimers than in the quatrimers, due to differences in their mass.

For oligomers with similar trans-/saturation content but different size, such as D2 and the oligomers higher than the pentamer discussed in an earlier publication (Li, S., L. Bouzidi, and S. S. Narine, Synthesis and Physical Properties of Triacylglycerol Oligomers: Examining the Physical Functionality Potential of Self-Metathesized Highly Unsaturated Vegetable Oils. Industrial & Engineering Chemistry Research (2013), the smaller oligomers start crystallizing at higher temperature (Tₒₙ^{C} (D2)=3.6° C. and Tₒₙ^{C} (Pentamer)=−16° C.) but complete crystallization at lower temperatures than larger oligomers (T_{off} (D2)=−55° C. and T_{off} (pentamer)=−30° C.). For the same saturation, the larger oligomers pack in less stable polymorphs, involve lower enthalpies of crystallization, and form more inhomogeneous phases than the smaller oligomers. One may note that for similar saturation values, the enthalpy of the larger oligomers are slightly smaller. The effect of size is indeed noticeable but not large enough to be more important than the effect of saturation.

### Melting Behavior

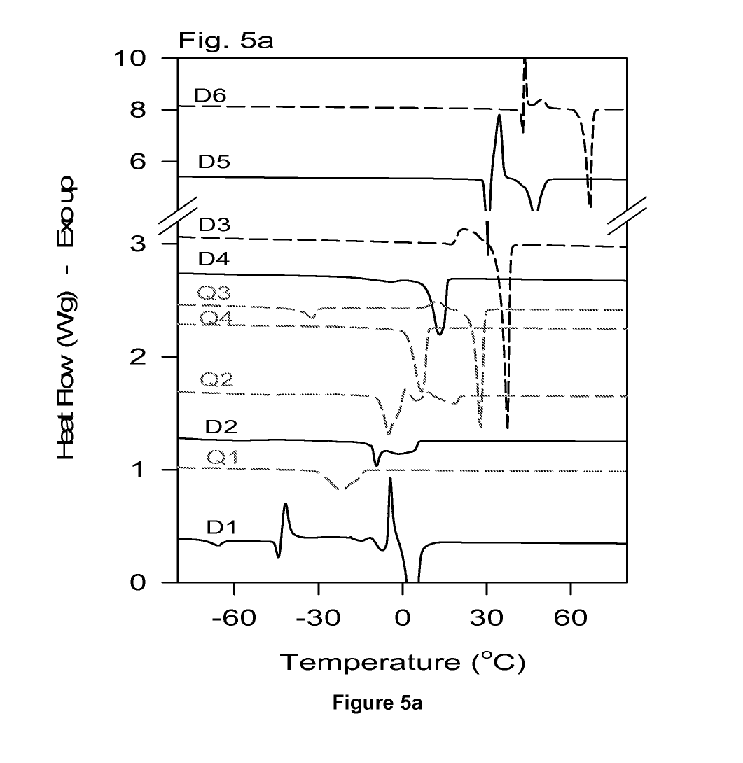

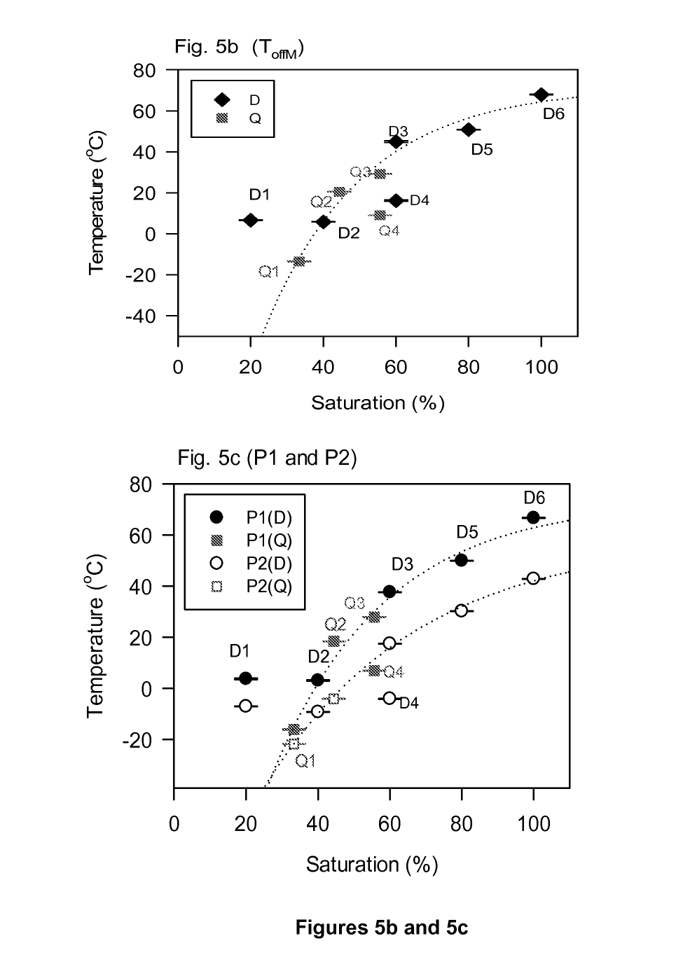

The DSC thermograms of the dimers and quatrimers obtained during heating at 5° C./min are presented in FIG. 5a. Except Q4 and D4 where only one and two melting transitions were detected, respectively, the dimers and quatrimers underwent several phase transitions during heating including recrystallization mediated by melt. Only one endotherm was present in the melting thermogram of Q4 indicating that a single phase was formed. The lower temperature exotherm of this compound was the manifestation of a further ordering or solid-solid transformation rather than the nucleation and growth of a second crystal phase. The leading endotherm of D4 (peak at −4° C. in FIG. 5a) is associated with the small and wide low temperature exotherm observed during crystallization (peak at −9° C. in FIG. 4a), and its main endotherm (peak at 13.4° C. in FIG. 5a) is associated with the two close high temperature exothermic events observed in its cooling thermogram (peaks at 12.9 and 11.0° C. in FIG. 4a). The first two endotherms observed in the heating cycles of D1, Q1, D2 and Q2 suggest the melting of two separate phases that coexisted in the solid state. These endotherms may be associated with successive exotherms observed in the corresponding cooling thermograms shown in FIG. 4a, and indicate that these compounds form multiphasic structures. Of course, the nature and relative content of the coexisting phases in each compound depend on the molecule's level of saturation.

The heating thermograms of D3, D5 and D6 started with the recording of the melting of the previously formed phases followed by strong recrystallization events and their subsequent melting. Note that the leading endotherm of D3 was weak and its following exotherm was wide indicating that although the phase that has nucleated was first driven by its saturated structural elements (leading peak in its cooling thermogram in FIG. 4a), it transformed to more stable but inhomogeneous crystal phases mainly by the reorganization of its unsaturated fatty acids (second peak in FIG. 4a). The sharp endotherms and recrystallization peaks observed in the heating thermograms of D5 and D6 are reminiscent of tristearin. Note however, that D6 presented two recrystallization peaks; whereas, D5 presented only one exotherm indicating the effect of the lone unsaturated oleic acid of D5 on the transformation path. Again, the extra steric hindrance prevented D5 to transform further in the melt.

The biphasic nature of solid Q3, as revealed by two separate exotherms of its cooling thermogram (curve Q3 in FIG. 4a), was confirmed by its melting thermogram (curve Q3 in FIG. 5a). Phase 1, melting at high temperature, is related to the packing influences of the saturated terminal fatty acids at one end, and phase 2, melting at a lower temperature, is related to the packing of the unsaturated terminal fatty acids at the other end. One may suggest that the onset of melting is primarily determined by the fusion of the solid at the unsaturated fatty acids linkages and the offset is determined mainly by the fusion at the saturated fatty chains.

The representative melting data of the dimers and quatrimers described herein shown in Table 9 below.

TABLE 9
Melting data of dimers and quatrimers obtained during the heating
rate at 3° C./min. Tonset, Toffset, Tp1, and Tp2 are the
temperature of onset, offset, Peak 1, and Peak 2, respectively

| | Melting | | | | |
|---|---|---|---|---|---|
| Samples | Tₒₙₛₑₜ (° C.) | T_{offset} (° C.) | T_{P1} (° C.) | Tₚ₂ (° C.) | Enthalpy (J/g) |
| D1 | −70.75 ± 0.20 | 6.65 ± 0.14 | 3.75 ± 0.23 | −6.98 ± 0.11 | 48.70 ± 2.00 |
| D2 | −17.00 ± 0.37 | 5.86 ± 0.06 | −1.11 ± 0.23 | −9.19 ± 0.06 | 48.87 ± 0.22 |
| D3 | 11.27 ± 1.21 | 44.99 ± 0.48 | 37.69 ± 0.03 | 7.50 ± 0.10 | 73.44 ± 6.92 |
| D4 | −19.84 ± 0.09 | 16.19 ± 0.37 | 13.58 ± 0.32 | −4.01 ± 0.14 | 67.58 ± 4.96 |
| D5 | 28.93 ± 0.03 | 50.83 ± 0.09 | 47.30 ± 0.11 | 30.29 ± 0.09 | 87.09 ± 5.93 |
| D6 | 42.06 ± 0.05 | 67.93 ± 0.19 | 66.74 ± 0.08 | 42.90 ± 0.05 | 138.40 ± 8.00 |
| Q1 | −29.91 ± 0.10 | −13.48 ± 0.10 | −16.06 ± 0.19 | −21.69 ± 0.01 | 35.06 ± 2.00 |
| Q2 | −8.02 ± 0.03 | 20.60 ± 0.03 | 18.39 ± 0.04 | −4.00 ± 0.03 | 42.56 ± 1.73 |
| Q3 | −37.32 ± 0.50 | 29.29 ± 0.12 | 27.96 ± 0.08 | −31.86 ± 0.09 | 61.98 ± 3.64 |
| Q4 | 2.11 ± 0.13 | 9.07 ± 0.03 | 6.94 ± 0.04 | | 53.13 ± 2.44 |

### Effect of Saturation Levels on Melting

The melting characteristic temperatures of the present oligomers were controlled by saturation and strongly affected by symmetry, similar to the crystallization characteristic temperatures. Ignoring for an instance the subtleties introduced by symmetry, one may see from FIG. 5a that all the characteristic temperatures (onset, offset and peak temperatures) increased, and the overall melting range decreased with increasing saturation. Without taking into account the least saturated dimer (D1) and the asymmetric D4 and Q4, the offset temperature of melting (FIG. 5b) and peak temperature of the last endotherm (P1 in FIG. 5c), presented an exponential rise to a maximum (R²>0.9930, dotted line in FIGS. 5b and 5c). These two parameters indicate the highest stability phase that may be formed in the compounds. The endotherm associated with the second most stable phase of the present oligomers (P2 in FIG. 5c), followed the same exponential trend as the endotherm that is associated with the most stable phase (P1 in FIG. 5c) indicating that the hierarchy in stability was globally preserved with increasing saturation. However, as may be seen in FIG. 5c, the difference in peak temperature between P1 and P2 increased with increasing saturation, denoting a differentiated effect of the saturated structural elements on the overall stability of the two phases. Furthermore, the enthalpy of P1 increased much more noticeably than P2 (FIG. 5a) indicating the growing preponderance of the high stability phase with increasing saturation.

### Effect of Symmetry and Molecular Size on Melting

The symmetry considerations about the center of the molecule and about the sn-2 positions of the glycerol backbones invoked to explain the differences in the crystallization behavior of oligomers with the same saturation levels may be invoked for the melting behavior. D1 and D2 exemplify the significance of symmetry in the melting behavior of the present oligomers. One may see that the last phase of D1 as represented by its melting trace (peak at ˜−9° C. in FIG. 5a), was similar to the phase presented by D2, despite a much lower level of saturation (20% compared to 40% in D2). This is attributed to the symmetry about the bridge of D1 that allowed a packing analogous to what has been allowed by twice % saturation mitigated by asymmetry in D2. This phase of D1 melted at even lower temperature than the phase presented by Q2 (peak at ˜−5° C. in FIG. 5a), a molecule that is not only asymmetrical but is also almost three times larger. This highlights an interplay between saturation, symmetry and molecular size. The balance between these structural elements is particularly delicate when differences in saturation are small.

The effect of symmetry about the center is manifest in the melting behavior of D3, which has its unsaturated fatty acids on one glycerol molecule and its saturated fatty acids on the other, and D4, which have its unsaturated and saturated fatty acids equally distributed on the glycerols. D3 started melting at a much higher temperature than D4 (11° C. compared to −20° C.) and recrystallized strongly, contrary to D4 that melted simply through two separate transitions. The same elements of symmetry considered in D3 and D4 motivated comparable differences in the melting behavior of Q3 and Q4. Q3, for example, also achieved its highest melting phase (peak at 28° C. in FIG. 5a) via a strong recrystallization contrary to Q4 that melted simply (peak at 7° C. in FIG. 5a). Similarly to crystallization, these differences are attributed to the proximity of the bridge in the trans-configuration and the stearic acids in D3(Q3), that may accommodate stronger contacts compared to D4(Q4) where the mixed distribution of the unsaturated and saturated fatty acids prevents the formation of more stable phases.

Note that Q3 presented a small leading endotherm at lower temperature (peak at −32° C. in FIG. 5a), indicating the melting of a low stability phase. This suggests that although the symmetry about the center of the molecules is a determining factor in the stability of the possible phases, the distribution of the fatty acid about the bridge is responsible for the added complexity to the transformation path of Q3 compared to Q4.

The differences observed in the effect of symmetry between the dimers and quatrimers were mitigated by molecular size. For example, the main endotherms of Q3 and Q4 were presented at 28 and 7° C., respectively, whereas, those of D3 and D4 were at the much higher temperature of 38 and 14° C., respectively. Also, the formation of two phases in D4 and one phase in Q4 is attributed to size differences (Q4 is twice as large as D4), wherein the necessary mass transfer for the nucleation of a second phase was enabled by D4 and not the much larger Q4. Note that the size of the oligomers manifested also with a reduction of the total enthalpy of melting similar to the enthalpy of crystallization; this is explainable by mass transfer limitations due to the larger size of the molecule.

### Effects of Saturation, Symmetry and Molecular Size—Trends

The synopsis of the overriding trends due to saturation, symmetry and molecular size on the thermal stability, and characteristic temperatures, range and enthalpy of the thermal transformations (crystallization and melting) occurring in the oligomers is presented in Table 10.

TABLE 10
Summary of the effects of saturation, symmetry and molecular size on the thermal
stability, and characteristic temperatures, range and enthalpy of the thermal transformations
observed during the crystallization and melting of the oligomers.

| | Thermal | Crystallization | | | Melting | | |
|---|---|---|---|---|---|---|---|
| | stability | Temperature | Range | Enthalpy | Temperature | Range | Enthalpy |
| Saturation | <20% | Increase | Decrease | Increase | Increase | Increase | Increase |
| | No Effect | | | | | | |
| | >20% | | | | | | |
| | Decrease | | | | | | |
| Symmetry | Decrease | Increase | Increase | No Effect | Increase | Increase | Increase |
| about the | | | | | | | |
| Center | | | | | | | |
| Symmetry | No Effect | Increase Complexity of | | No Effect | Increase Polymorphism | | |
| about the | | the Crystallization Path | | | | | |
| Bridge | | | | | | | |
| Size | No Effect | Decrease | Decrease | Decrease | Decrease | Increase | Increase |

Six dimers and four quatrimers with controlled saturation and trans-configurations, and having different terminal structures were synthesized from oleic or stearic acid derivatives. The targeted structures were confirmed by ¹H-NMR and ¹³C-NMR as well as MS. The thermal stability and thermal transitions data of the oligomers obtained by TGA and DSC showed that the relative number of straight fatty chains was the best structural variable for monitoring structure—physical property relationships. This variable, referred to as the level of saturation, or simply saturation, was found to be the overriding driver of the phase behavior of the oligomers. However, similar to TAGs, positional isomerism and size played a significant role in determining the crystallization and melting behavior. Note that, although the effect of size is indeed noticeable, it is not strong enough to be more important than the effect of saturation.

The thermal stability of the dimers was mainly affected by the degree of unsaturation and slightly by the relative position of the unsaturated fatty acids. The decomposition temperatures increased from the most saturated to the most unsaturated dimers and quatrimers. It was demonstrated that unsaturated fatty chains imparts strength to their closest weakest links, i.e., the β-hydrogens at the sn-1(3) position, measurably enhancing the thermal stability. Despite the differences in the degradation profiles that are due to the differences in the structures, the thermal degradation data indicated very good thermal stability for all the oligomers of this effort, better than common commercial vegetable oils.

The effect of saturation on the thermal behavior of the dimers and quatrimers manifested notably in the DSC thermograms. The differences in saturation levels produced variations in the number, extent and magnitude of the recorded thermal transitions. The thermal parameters of the dimers, quatrimers as well as of the oligomers of our previous work (Li, S., L. Bouzidi, and S. S. Narine, Synthesis and Physical Properties of Triacylglycerol Oligomers: Examining the Physical Functionality Potential of Self-Metathesized Highly Unsaturated Vegetable Oils. Industrial & Engineering Chemistry Research (2013), all adhere very well to predictive trends. Barring the effect of symmetry and size, the structure-function relationships were found to adhere well to predictive trends. The onset of crystallization of the oligomers increased almost linearly with increasing saturation from −22° C. for the least saturated to 40° C. for the most saturated oligomer, and the offset temperature of crystallization increased exponentially from −77° C. to 38° C. leading to increasingly shorter crystallization spans. The peak temperatures of crystallization of the oligomers also increased exponentially with increasing saturation. The least saturated dimer completed its crystallization over ˜55° C.; whereas, the most saturated crystallized in a temperature window of −2° C.

The crystallization and melting data suggested the competition of two different transformation processes, one that is established mainly by the trans- and saturated structural elements, and another that is driven by the unsaturated fatty acids of the oligomer. The data indicated that the disrupting effect of the unsaturated chains is minimized as saturation increases leading to polymorphic transformations being more likely than the nucleation of new phases.

The notable role of positional isomerism and size in the thermal behavior of the oligomers was also revealed. The strong effect of symmetry on the way these compounds organize into solid phases was evidenced by large differences in the crystallization and melting parameters of similarly saturated compounds. Differences of ˜10° C. and 30° C. were recorded in the onset of crystallization and offset of melting, respectively, between dimers of the same saturation but different symmetry. In the larger sized quatrimers, these difference were ˜6° C. and 20° C., respectively. For the same saturation levels, the larger oligomers pack in less stable and much more inhomogeneous phases than the smaller oligomers.

This document showed that the thermal parameters of TAG oligomers may be adjusted in a very broad range by saturation content, position of the fatty acids and oligomer size. The extent to which the crystallization and melting paths may be controlled by varying the degree of saturation was remarkably wide-ranging and bodes well for the custom engineering of a large variety of usages. Furthermore, the findings motivate the prospect of using safe and non-toxic metathesis routes for the development of easily custom designed economical bio-based materials, which include but are not limited to, waxes, base stocks for lubricant applications or a base stock blend component for use in a finished lubricant, and crystallization depressant additives and/or crystal size reduction additives for biodiesel.

The foregoing detailed description and accompanying figures have been provided by way of explanation and illustration, and are not intended to limit the scope of the invention. Many variations in the present embodiments illustrated herein will be apparent to one of ordinary skill in the art, and remain within the scope of the invention and their equivalents. The skilled person in the art will recognize many variations that are within the spirit of the invention and scope of any current or future claims.

What is claimed is:

1. A triacylglycerol dimer composition comprising the following structure: 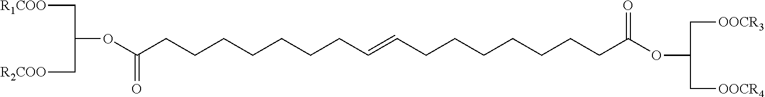
wherein R₁COO, R₂COO, R₃COO, and R₄COO are independently either an oleic acid anion or a stearic acid anion.

2. The composition of claim 1, wherein the triacylglycerol dimer comprises bis(1,3-bis(oleoyloxy)propan-2-yl)octadec-9-enedioate.

3. The composition of claim 1, wherein the triacylglycerol dimer comprises 1-(1,3-bis(oleoyloxy)propan-2-yl) 18-(1-(oleoyloxy)-3-(stearoyloxy)propan-2-yl)octadec-9-enedioate.

4. The composition of claim 1, wherein the triacylglycerol dimer comprises 1-(1,3-bis(oleoyloxy)propan-2-yl) 18-(1,3-bis(stearoyloxy)propan-2-yl)octadec-9-enedioate.

5. The composition of claim 1, wherein the triacylglycerol dimer comprises 1-(1-(oleoyloxy)-3-(stearoyloxy)propan-2-yl) 18-(1-(oleoyloxy)-3-(stearoyloxy)propan-2-yl)octadec-9-enedioate.

6. The composition of claim 1, wherein the triacylglycerol dimer comprises 1-(1,3-bis(stearoyloxy)propan-2-yl) 18-(1-(oleoyloxy)-3-(stearoyloxy)propan-2-yl)octadec-9-enedioate.

7. The composition of claim 1, wherein the triacylglycerol dimer comprises bis(1,3-bis(stearoyloxy)propan-2-yl)octadec-9-enedioate.

8. The composition of claim 1, wherein the triacylglycerol dimer comprises an onset temperature of thermal degradation in a range of about 325° C. to about 385° C.

9. The composition of claim 1, wherein the triacylglycerol dimer comprises a peak temperature of the main DTG peak in a range of about 375° C. to about 420° C.

10. The composition of claim 1, wherein the triacylglycerol dimer comprises: (i) a crystallization onset temperature in a range of about −22° C. to about 40° C.; (ii) a crystallization offset temperature in a range of about −77° C. to about 38° C.; and (iii) an enthalpy of crystallization in a range of about 35 J/g to about 155 J/g.

11. The composition of claim 1, wherein the triacylglycerol dimer comprises: (i) a melting onset temperature in a range of about −71° C. to about 42° C.; (ii) a melting offset temperature in a range of about 5° C. to about 68° C.; and (iii) an enthalpy of melting in a range of about 46 J/g to about 147 J/g.

12. A triacylglycerol quatrimer composition comprising the following structure: 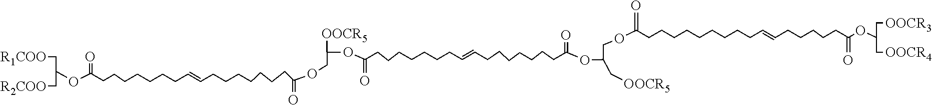
wherein R₁COO, R₂COO, R₃COO, R₄COO, and R₅COO are independently either an oleic acid anion or a stearic acid anion.

13. The composition of claim 12, wherein the triacylglycerol quatrimer comprises bis(1,3-bis(oleoyloxy)propan-2-yl) O′¹,O¹-(((octadec-9-enedioyl)bis(oxy))bis(3-(oleoyloxy)propane-2,1-diyl))bis(octadec-9-enedioate).

14. The composition of claim 12, wherein the triacylglycerol quatrimer comprises 1-(2,23-bis((oleoyloxy)methyl)-4,21,26,43,48-pentaoxo-45-((stearoyloxy)methyl)-3,22,25,44,47-pentaoxapentahexaconta-12,34,56-trien-1-yl) 18-(1,3-bis(oleoyloxy)propan-2-yl)octadec-9-enedioate.

15. The composition of claim 12, wherein the triacylglycerol quatrimer comprises 1-(2,23-bis((oleoyloxy)methyl)-4,21,26,43,48-pentaoxo-45-((stearoyloxy)methyl)-3,22,25,44,47-pentaoxapentahexaconta-12,34-dien-1-yl) 18-(1,3-bis(oleoyloxy)propan-2-yl)octadec-9-enedioate.

16. The composition of claim 12, wherein the triacylglycerol quatrimer comprises bis(1-(oleoyloxy)-3-(stearoyloxy)propan-2-yl) O′¹,O¹-(((octadec-9-enedioyl)bis(oxy))bis(3-(oleoyloxy)propane-2,1-diyl))bis(octadec-9-enedioate).

17. The composition of claim 12, wherein the triacylglycerol quatrimer comprises an onset temperature of thermal degradation in a range of about 375° C. to about 385° C.

18. The composition of claim 12, wherein the triacylglycerol quatrimer comprises a peak temperature of the main DTG peak in a range of about 415° C. to about 425° C.

19. The composition of claim 12, wherein the triacylglycerol quatrimer comprises: (i) a crystallization onset temperature in a range of about −21° C. to about 11° C.; (ii) a crystallization offset temperature in a range of about −48° C. to about −16° C.; and (iii) an enthalpy of crystallization in a range of about 35 J/g to about 62 J/g.

20. The composition of claim 12, wherein the triacylglycerol quatrimer comprises: (i) a melting onset temperature in a range of about −38° C. to about 2° C.; (ii) a melting offset temperature in a range of about −14° to about 30° C.; and (iii) an enthalpy of melting in a range of about 33 J/g to about 65 J/g.
